(19) United States
(12) Patent Application Publication
(10) Pub. No.: US 2007/0092509 A1
(43) Pub. Date: Apr. 26, 2007

(54) Method for ex-vivo separation of apoptotic chromatin fragments from blood or plasma for prevention and treatment of diverse human diseases

(75) Inventors: Indraneel Mittra, Bophal (IN); Urmila Chandrashekhar Samant, Mumbai (IN); Gopesh Kumar Modi, Bhopal (IN); Pradyumna Kumar Mishra, Bhopal (IN); Gobichettipalayam Subbaratnam Bhuvaneshwar, Trivandrum (IN)

Correspondence Address: MERCHANT & GOULD PC, P.O. BOX 2903, MINNEAPOLIS, MN, 55402-0903, US

(73) Assignee: Tata Memorial Centre, Mumbai (IN)

(21) Appl. No.: 11/588,446

(22) Filed: Oct. 27, 2006

Related U.S. Application Data
(63) Continuation-in-part of application No. PCT/IN05/00353, filed on Oct. 20, 2005.

(51) Int. Cl. A61K 48/00; A61M 31/00

(52) U.S. Cl. 424/140.1; 604/500

(57) ABSTRACT

A method of prevention/treatment of pathological consequences of DNA damage triggered by incorporation of circulating apoptotic chromatin fragments into healthy cells of individuals/patients in need therefore, said method comprising ex vivo or extra corporeal treatment of blood/plasma for removal of circulating chromatin fragments released from apoptotic cells which apoptotic chromatin fragments are capable of triggering DNA damage leading to genomic instability, senescence, apoptosis and cancerous transformation of healthy cells on being integrated into their genomes

The patent ot application file contains at least one drawing executed in coler. Copies of this patent or patent application with color drawing(s) will be provided by the Office upon request and payment of the necessary fee.

### FIELD OF THE INVENTION

The present invention relates to method of prevention/treatment of pathological consequences of DNA damage triggered by incorporation of circulating apoptotic chromatin fragments into healthy cells of individuals/patients in need therefore, said method comprising ex vivo or extra corporeal treatment of blood/plasma for removal of circulating chromatin fragments released from apoptotic cells which apoptotic chromatin fragments are capable of triggering DNA damage leading to genomic instability, senescence, apoptosis and cancerous transformation of healthy cells on being integrated into their genomes. Said method of prevention/treatment may be carried out in a system for ex-vivo or extra corporeal treatment of blood to prevent pathological consequences arising from circulating chromatin fragments derived from apoptotic cells being ingested by healthy somatic cells. More particularly, the present invention relates to method of prevention/treatment comprising ex-vivo or extra corporeal treatment of blood to prevent pathological consequences such as DNA damage, genomic instability, senescence, apoptosis and oncogenic (cancerous) transformation in healthy somatic cells resulting from ingestion of circulating apoptotic chromatin fragments that are present in the blood of normal subjects and in higher quantities in patients with various diseases. These diseases may include cancer, atherovascular diseases, diabetes, Alzheimer's disease, Parkinson's disease, stroke, severe infections, sepsis, renal failure, HIV/AIDS, autoimmune disorders etc. as well as ageing and other age related disorders. The removal of apoptotic chromatin fragments from blood may be effected by a combination of separating means comprising adsorption with antibodies, cationic resin like DEAE—Sephadex, filtration, centrifugation and principles of flowcytometry-assisted cell sorting.

### BACKGROUND AND PRIOR ART

Active cellular suicide or programmed cell death, also known as apoptosis, plays an important role in animal development, tissue homeostasis, immune response and a wide variety of pathological conditions including cancer, atherovascular diseases, diabetes, Alzheimer's disease, Parkinson's disease, stroke, severe infections, sepsis, renal failure, HIV/AIDS, autoimmune disorders etc. [Wyllie, A. H., Kerr, J. F. R., Currie, A. R. Cell death: the significance of apoptosis. Int. Rev. Cytol 68, 251-306 (1980); Fadeel, B., Orrenius, S., Zhivotovsky, B. Apoptosis in human disease: a new skin for the old ceremony. Biochem. Biophys. Res. Com. 266, 699-717 (1999)]. Apoptosis is characterized by programmed or systematic activation of a number of genes, especially those coding for caspases, which lead to cleavage of the chromatin/DNA into smaller fragments which are entrapped in apoptotic bodies that result from disintegration of the apoptotic cells. Under physiological conditions these apoptotic bodies and the chromatin/DNA contained within them are efficiently removed when ingested by macrophages, also known as “professional phagocytes”. However, apoptotic bodies can also be ingested by non-macrophage cells or “non-professional phagocytes”, such as fibroblasts, which are incapable of efficiently clearing them from the body. [Parnaik, R., Raff, M. C. & Scholes, J. Differences between the clearance of apoptotic cells by professional and non-professional phagocytes. Curr. Biol. 10, 857-860 (2000)]. When ingested by macrophages, the engulfed chromatin/DNA is known to be degraded and ultimately lost with the death of the scavenging cells. However, the fate of non-macrophage cells after they engulf the apoptotic chromatin fragments remains largely unknown.

Hundreds of billions of cells die in the body everyday and an equal number of cells are generated to replace them [Fliedner T. M., Graessle D, Paulsen C. & Reimers K. Structure and functions of bone marrow hemopoiesis : Mechanisms of response to ionizing radiation exposure. Cancer Biotherapy & Radio pharmaceuticals 17, 405-425 (2002)] Unless these apoptotic cells are efficiently eliminated by phagocytosis, apoptotic chromatin/DNA can enter the blood stream from tissues and blood cells undergoing normal apoptotic turnover. Indeed, with the recent availability of a quantitative sandwich-enzyme-immunoassay which employs antibodies to both DNA and histones (Cell Death Detection ELISA Plus, Roche Biochemicals), fragments of chromatin in the form of mono- and oligonucleosomes have been shown to be present in sera of normal persons, and in higher quantities in patients with cancer, systemic lupus erythematosus, inflammation, sepsis, cerebral stroke etc. indicating a higher level of ongoing apoptosis is these conditions. [Holdenrieder, S. et al. Circulating nucleosomes in serum. Ann. N Y Acad Sci. 945, 93-102 (2001); Williams, R. C., Malone, C. C., Meyers, C., Decker, P., Muller, S. Detection of nucleosome particles in serum and plasma from patients with systemic lupus erythematosus using monoclonal antibody 4H7. J Rheumatol 28, 81-94 (2001); Zeerleder S et al. Elevated nucleosome levels in systemic inflammation and sepsis. Crit. Care Med. 31, 1947-1951 (2003); Geiger S et al. Nucleosomes in serum of patients with early cerebral stroke. Cerebrovasc. Dis. 21, 32-37 (2006)].

It has been demonstrated that in patients with cancer, the elevated basal level of circulating chromatin rises further following chemotherapy or radiotherapy within 24-72 hours [Holdenrieder, S. et al. Nucleosomes in serum of patients with benign and malignant diseases. Int J Cancer 95, 114-120 (2001)].

Blood component therapy/transfusion is a common therapeutic procedure. Since apoptotic chromatin fragments are known to circulate in blood of normal individuals, it is possible that during transfusion of blood or blood products such apoptotic chromatin fragments are transferred to the recipient leading to an increase in circulating chromatin burden.

The genome of a cancer cell is dynamically unstable. Genomic/chromosomal instability is the hallmark of cancer, and has been shown to precede cancerous transformation in several systems examined with the implication that it might be the cause rather than consequence of malignancy [Stoler, D. L. et al. The onset and extent of chromosomal instability in sporadic colorectal tumor progression. Proc. Natl. Acad. Sci. 96, 15121-15126 (1999)]. Presently, the nature of triggering events that precipitate genomic instability is unknown.

There have been a few recent reports which show that transfer of DNA can occur horizontally when apoptotic cells are co-cultivated with a variety of recipients in vitro. When apoptotic transformed lymphoid cells carrying Epstein-Barr virus (EBV) are co-cultivated with either human fibroblasts or macrophages, or bovine endothelial cells, expression of EBV encoded genes can be detected in the recipient cells. Fluorescence in situ hybridization (FISH) analysis showed uptake of human DNA as well as integrated EBV-DNA into the nuclei of bovine endothelial cells [Holmgren, L. et al. Horizontal transfer of DNA by the uptake of apoptotic bodies. Blood 93, 3956-3963 (1999)]. In another study it is demonstrated that prostate cancer cells exchange drug resistance genes in vitro through engulfment of apoptotic bodies. [de la Taille A., Chen, M. W., Burchardt, M., Chopin, D. K. & Buttyan R. Apoptotic conversion: evidence for exchange of genetic information between prostate cancer cells mediated by apoptosis. Cancer Res. 59, 5461-5463 (1999)]. However, these studies did not investigate whether the horizontally transferred apoptotic bodies induce DNA damage, genomic instability, senescence, apoptosis and/or malignant transformation in the recipient cells.

Two studies have provided evidence for spread of viruses via apoptotic cells. One of these studies demonstrated that HIV-1 DNA may be transferred from one cell to another by uptake of apoptotic bodies by a mechanism which is independent of binding of the virus to the CD4 receptor [Spetz, A., Patterson, B. K., Lore, K., Andersson, J., and Holmgren, L. Functional gene transfer of HIV DNA by an HIV receptor-independent mechanism. J Immunol 163, 736-742 (1999)]. In the other study, the investigators while working on a way to enhance the spread of adenoviral vectors—the delivery vehicles for gene therapy—found that induction of apoptosis after onset of viral DNA replication enhanced the spread of the virus among cervical cancer cells in vitro [Mi, J., Li, Z. Y., Ni, S., Steinwaerder, D., Lieber, A. Induced apoptosis supports spread of adenovirus vectors in tumors. Hum. Gene Ther. 12, 1343-1352 (2001)].

While the above studies showed that genetic or viral DNA transfer can occur horizontally via apoptotic bodies, only one study has investigated as to whether the transfer of DNA can lead to oncogenic transformation of the recipient cells [Bergsmedh, A. et al. Horizontal transfer of oncogenes by uptake of apoptotic bodies. Proc. Natl. Acad. Sci 98, 6407-6411 (2001)]. In that study apoptotic normal rat fibroblast cells or those that had been transfected with H-rasV12 and human myc oncogenes were co-cultured with either normal mouse fibroblast cells or mouse fibroblast cells that had a p53 −/− genotype. Transformed colonies were obtained provided both the donor apoptotic cells and the recipient cells had been genetically engineered, i.e. only when H-rasV12 and human myc transfected rat fibroblast were used as apoptotic donors and the recipient mouse fibroblast cells were p53 −/−. No foci were observed if normal rat fibroblast cells were used as apoptotic donors or when normal mouse fibroblast cells were used as recipients. Since both the recipient and donor cells had to be specifically genetically engineered to produce cancerous transformation, it is not at all obvious from this study whether oncogenic transformation by apoptotic cells/bodies can occur in normal somatic cells under natural physiological conditions.

However, it must be pointed out that none of the above studies have investigated as to whether apoptotic chromatin fragments, that circulate in blood of healthy subjects, and in higher quantities in patients suffering from various diseases, can enter the normal somatic cells in the body, get integrated in their genomes and bring about harmful pathological consequences.

The cause of cancer is unknown and the results of current treatments of the disease are far from satisfactory. In spite of many refinements in techniques of surgery and radiotherapy and the use of numerous newly developed chemotherapeutic and biological agents, there has not been any marked reduction in mortality from common adult cancers [Bailar III, J. C. & Gornik, H. L. Cancer undefeated. N. Eng. J. Med. 336,1569-1574 (1997)]. Revolutionary approaches involving a conceptual shift from current treatment practices are, therefore, needed.

There is a large body of literature on the potential treatment of cancer based on enhancing or promoting apoptosis in tumors by manipulating apoptosis related genes, receptors or other molecular pathways of the cell death machinery [see for review: Nicholson, D. W. From bench to clinic with apoptosis-based therapeutic agents. Nature. 407, 810-816 (2000)]. Numerous approaches are currently being pursued to discover specific drug targets through which apoptosis could be enhanced [Zhang, J. Y. Apoptosis-based anticancer drugs. Nature Reviews Drug Discovery 1, 101-102 (2002)]; [Los, M. et al. Anticancer drugs of tomorrow: apoptotic pathways as targets for drug design. Drug Discov Today. 8, 67-77 (2003); Brachat, A. et al. A microarry-based, integrated approach to identify novel regulators of cancer drug response and apoptotis. Oncogene. 21, 8361-71 (2003)]. In fact, the traditional therapeutic modalities for cancer, namely chemotherapy and radiotherapy, are founded on the principle of destroying cancer cells by the induction of apoptosis [Chu, E., DeVita, V. T. Principles of cancer management: chemotherapy. In Cancer: Principles and Practice of Oncology, 6th Edition, DeVita, V. T., Hellman, S., Rosenberg, S. A. Lippincott Williams & Wilkins, Philadelphia. pp. 289-306, 2001]. Thus, the above traditional approaches to cancer therapy greatly increase the apoptotic chromatin burden in the body. Indeed, it is now established that apoptotic chromatin fragments from the tumor cells are released into the circulation after chemotherapy and/or radiotherapy thereby increasing the chromatin burden in blood. [Holdenrieder, S. et al. Nucleosomes in serum of patients with benign and malignant diseases. Int J Cancer 95, 114-120 (2001)].

Progressive DNA damage leading to genomic instability, senescence and apoptosis of cells underlies human ageing [Kirkwood T. B. L. Understanding the odd science of aging. Cell 120, 437-447 (2005)]. Although free radicals generated within the body have been implicated as the DNA damaging agent related to ageing, this theory has not been satisfactorily substantiated [Lombard D. B. et al. DNA repair, genome stability, and aging Cell 120, 497-512 (2005)]. Enhanced apoptosis resulting from DNA damage is also associated with a wide variety of age related degenerative diseases such as Alzheimer's disease, Parkinson's disease, Stroke, Atherovascular diseases, Diabetes etc. [Jellinger K. A. Cell death mechanisms in neurodegeneration. J Cell Mol Med 5, 1-17 (2001); Bennett M. R. Apoptosis in the cardiovascular system. Heart 87, 480-487 (2002) ; Otton R, Soriano F G, Verlengia R, Curi R. Diabetes induces apoptosis in lymphocytes. J Endocrinol 182, 145-56 (2004)].

Increased cellular apoptosis is also associated with inflammatory processes such as infections, sepsis and sepsis syndrome, multi-organ system failure as well as autoimmune disorders [Hotchkiss R S et al. Apoptotic cell death in patients with sepsis, shock, and multiple organ dysfunction. Crit Care Med. 27, 1230-1251 (1999); Apoptosis and Autoimmunity from Mechanisms to Treatment, Edited by J. R. Kalden and M. Herrmann. Co. Wiley-Vch, Weinheim (2003);]. The above conditions are also known to be associated with high circulating levels of apoptotic chromatin fragments in blood. [Zeerleder S et al. Elevated nucleosome levels in systemic inflammation and sepsis. Crit. Care Med. 31, 1947-1951 (2003); Williams, R. C., Malone, C. C., Meyers, C., Decker, P., Muller, S. Detection of nucleosome particles in serum and plasma from patients with systemic lupus erythematosus using monoclonal antibody 4H7. J Rheumatol 28, 81-94 (2001)]. It has been reported that renal failure is associated with an increased apoptotic turnover which may contribute to the high mortality in this condition. [D'Intini V et. al. Longitudinal study of apoptosis in chronic uremic patients. Semin Dial, 16, 467-73 (2003) ; U.S. Renal Data System, USRDS 2005 Annual Data Report: Atlas of End-Stage Renal Disease in the United States, National Institute of Diabetes and Digestive and Kidney Diseases, Bethesda, MD, 2006]. HIV infection/AIDS is also associated with extremely high apoptotic turnover in CD4 positive cells and is causally related to the multiple pathological consequences/complications of this disease. [Badley A. d, Pilon A. A., Landay A & Lynch D. H. Mechanisms of HIV-associated lymphocyte apoptosis. Blood, 96, 2951-2964 (2000)]

Blood and blood products, that are routinely transfused for diverse medical indications, are known to be associated with an array of adverse consequences [Dellinger EP, Anaya D. A Infectious and immunologic consequences of blood transfusion. Critical Care 8, S18-S23 (2004)]. Transfusion of blood or blood products can increase the apoptotic chromatin burden in the recipient by i) delivering the existing apoptotic chromatin in the donor blood/blood products, ii) delivering apoptotic chromatin fragments that are derived from cells that undergo apoptosis during storage and processing. This chromatin overload may have deleterious effects on the recipient. Despite numerous medical advances there are no satisfactory treatments available for most of the above conditions. There is no teaching that integration of apoptotic chromatin fragments with healthy cells may lead to DNA damage, genomic instability, senescence, apoptosis and cancerous transformation or that these genomic/cellular changes may lead to age related degenerative diseases, transformation of healthy cells to cancerous cells, the spread of cancer within the body, pathological consequences associated with severe infections, sepsis syndrome, multi-organ failure, HIV/AIDS, renal failure, autoimmune disorders etc. There may be a need for removal of circulating apoptotic chromatin fragments to prevent their integration into healthy cells as a method of treatment for prevention/retardation of the process of initiation and spread of cancer in the body, age related degenerative diseases and perhaps to ageing itself, pathological consequences associated with severe infections, renal and auto-immune diseases as well as adverse effects related to blood transfusion.

### OBJECT OF THE PRESENT INVENTION

Thus, the principal object of the present invention is to provide a method of prevention/treatment of pathological consequences of DNA damage triggered by incorporation of circulating apoptotic chromatin fragments into healthy cells of individuals/patients in need therefore, said method comprising ex vivo or extra corporeal treatment of blood/plasma for removal of circulating chromatin fragments released from apoptotic cells which apoptotic chromatin fragments are capable of triggering DNA damage leading to genomic instability, senescence, apoptosis and cancerous transformation of healthy cells on being integrated into their genomes.

A further object of the present invention is to provide a method of treatment where the extra corporeal treatment of blood is carried out in a system using separating means employing agents selected from chemical agents (adsorption), or immunological agents/antibodies (adsorption), or biochemical agents/enzymes (degradation); or contraption adapted for centrifugation of plasma to precipitate chromatin fragments; or filtration system having appropriate porosity adapted to filtration of plasma or blood; or use flowcytometry-assisted sorter-based methods for separation of chromatin. Another object of the present invention is to provide a method of treatment of blood in order to prevent the initiation and spread of cancer in the body; prevent or retard the process of ageing and age related diseases such as Alzheimer's disease, Parkinson's disease, stroke, atherovascular diseases, diabetes as well as renal failure, infections, sepsis syndrome, multiorgan failure, autoimmune disorders, HIV/AIDS etc.; prevent the spread of viral infection in the body and prevent harmful effects of transfusion of blood or blood products.

### SUMMARY OF THE INVENTION

Thus, according to the main aspect of the present invention there is provided method of prevention/treatment of pathological consequences of DNA damage triggered by incorporation of circulating apoptotic chromatin fragments into healthy cells of individuals/patients in need therefore, said method comprising ex vivo or extra corporeal treatment of blood/plasma for removal of circulating chromatin fragments released from apoptotic cells which apoptotic chromatin fragments are capable of triggering DNA damage leading to genomic instability, senescence, apoptosis and cancerous transformation of healthy cells on being integrated into their genomes.

According to another aspect there is provided method of treatment where the extra corporeal treatment of blood is carried out in a system for ex vivo or extra corporeal treatment of blood or plasma for removal of chromatin fragments released from apoptotic cells, said chromatin fragments being capable of getting integrated into the genomes of healthy cells as well as triggering DNA damage genomic instability, senescence, apoptosis and oncogenic transformation, said system comprising:

means adapted for removal of plasma containing apoptotic chromatin fragments from blood cells; separating means adapted to remove apoptotic chromatin fragments from the said plasma;

means adapted to reconstitute blood after removal of the apoptotic chromatin fragments from the said plasma;

means adapted to communicating and guiding of blood or plasma from body to the said separating means through the means for removal of plasma containing apoptotic chromatin fragments from blood cells, to means to reconstitute blood and direct the treated blood back into the body.

### DETAILED DESCRIPTION OF THE INVENTION

The present inventors have found that apoptotic chromatin fragments purified from blood of healthy subjects when added repeatedly every alternate day to recipient cells in culture are ingested by them which then induce DNA damage leading to genomic instability, senescence and apoptosis in them.

These in vitro findings are similar to cellular changes that are seen in human ageing. However, it must be pointed out that the potency of apoptotic chromatin fragments derived from normal subjects is far lower than those derived from cancerous subjects, especially those derived after chemo- or radiotherapy. Nevertheless, apoptotic chromatin fragments purified from as little a 100-μl of serum from healthy subjects when applied repeatedly are capable of inducing the above pathological changes. These findings, therefore, suggest that continuous and repeated exposure of healthy cells in the body to circulating apoptotic chromatin fragments throughout life may cause progressive damage to their DNA resulting in genomic instability, senescence and apoptosis of these cells. This process may contribute to a wide range of age related degenerative diseases mentioned above. In fact, the process of progressive DNA damage caused by circulating apoptotic chromatin fragments may underlie the phenomenon of ageing itself. Accordingly, the present invention provides for a conceptual shift from the existing etiological theories and potential therapeutic approaches to age related degenerative diseases and perhaps to ageing itself.

Therefore, a therapeutic method which removes apoptotic chromatin fragments ex-vivo or extra corporeally from blood may prevent or retard ageing and its associated degenerative diseases.

The present studies described herein show that some cultured cells that have undergone senescence, i.e. prolonged growth arrest, by repeated addition of purified apoptotic chromatin fragments derived from blood of healthy subjects as described above get re-activated after several days in culture and are transformed into cancerous cells. This finding suggests that circulating apoptotic chromatin fragments by attacking healthy cells repeatedly throughout life may underlie the process of initiation of cancer in the body.

Therefore, ex vivo or extra corporeal removal of apoptotic chromatin fragments from blood may act as a method of treatment/prevent/retard the process of initiation of cancer in the body.

The studies described herein also show that a single addition of purified apoptotic chromatin fragments derived from as little as 5-100 μl of serum from cancer patients, who have been recently treated with chemo- or radiotherapy, to recipient cells in culture are ingested by the recipients which then induce DNA damage, genomic instability and oncogenic transformation in them.

Thus, the current therapeutic modalities for cancer may be counterproductive since the apoptotic chromatin fragments released from tumor cells following chemotherapy and/or radiotherapy may actually promote spread of cancer in the body by being ingested by healthy cells.

Therefore, ex-vivo or extra corporeal removal of apoptotic chromatin fragments from blood of cancer patients following chemo- and/or radiotherapy may prevent or retard the spread of cancer within the body.

The studies described herein also show that when apoptotic chromatin fragments purified from plasma/serum of patients with diabetes, renal failure and sepsis are added to lymphocytes isolated from healthy individuals, the chromatin fragments are internalized and the lymphocytes undergo apoptosis.

These findings suggest that, since apoptosis is one of the biological endpoints of DNA damage, ex vivo extra corporeal removal of apoptotic chromatin fragments from patients suffering from diabetes, renal failure and sepsis may prevent or retard the pathological consequences of these diseases.

Since transfusion of blood also adds to increase the load of apoptotic chromatin fragments in the body, a method for ex vivo removal of apoptotic chromatin from blood or blood products before transfusion may help to prevent their adverse effects.

Thus, the results of the above studies suggest that the uptake of circulating apoptotic chromatin may underlie the process of ageing, age related and degenerative conditions such as Alzheimer's disease, Parkinson's disease, stroke, atherovascular diseases, diabetes etc.

The above findings also suggest that the uptake of circulating chromatin may underlie the process of cancer initiation in the body. The same process may also underlie spread of cancer from the site of the primary disease since chromatin fragments released from apoptotic cancerous cells, especially after chemo- or radiotherapy, and carried in circulation are likely to infect healthy cells in other parts of the body.

The same process may also underlie the pathological consequences associated with severe infections, sepsis syndrome, multi-organ failure, HIV/AIDS, renal failure, autoimmune disorders, etc. which are associated with increased apoptosis and consequently increased apoptotic chromatin burden in circulation. Thus, it follows that if such fragmented circulating chromatin particles from apoptotic cells can be prevented from being ingested by healthy cells in the body it could form a method of treatment/prevention/retardation of pathological conditions, such as initiation and spread of cancer, age related and degenerative conditions such as Alzheimer's disease, Parkinson's disease, stroke, atherovascular diseases, diabetes as well as infections, sepsis syndrome, multiorgan failure, autoimmune disorders, HIV/AIDS, renal failure etc.

Finally, the prevention of circulating apoptotic chromatin from attacking healthy cells may retard the process of aging.

Hence a method of treatment has been envisaged to prevent the initiation and spread of cancer in the body; prevent or retard the process of ageing and age related diseases such as Alzheimer's disease, Parkinson's disease, stroke, atherovascular diseases, diabetes as well as renal failure, infections, sepsis syndrome, multiorgan failure, autoimmune disorders, HIV/AIDS etc.; prevent the spread of viral infection in the body and prevent harmful effects of transfusion of blood or blood products and prevent/treat all other diseases associated with increased apoptosis. The method comprises ex vivo or extra corporeal purification of blood such that the circulating apoptotic chromatin fragments are removed from the blood so that they are not allowed to attack healthy cells to be incorporated in their genome and damage/transform these cells.

Such method of treatment comprising ex vivo or extra corporeal purification of blood that is carried out using the system/device of the invention is capable of being used to prevent the initiation and spread of cancer in the body; prevent or retard the process of ageing and age related diseases such as Alzheimer's disease, Parkinson's disease, stroke, atherovascular diseases, diabetes as well as renal failure, infections, sepsis syndrome, multiorgan failure, autoimmune disorders, HIV/AIDS etc.; prevent the spread of viral infection in the body and prevent harmful effects of transfusion of blood or blood products and prevent/treat all other diseases associated with increased apoptosis The removal of chromatin fragments derived from apoptotic cells in circulation in blood can be achieved by employing a system for treatment of blood where selective separating means employing chemical, immunological or enzymatic agents; centrifugation; filtration; or flowcytometry-assisted cell sorter like processes are used. The removal of chromatin fragments from circulation may prevent their integration into recipient cell genomes and its subsequent pathological consequences. The system according to the invention is adapted to remove ex vivo such apoptotic chromatin fragments assisting in the treatment of the disease conditions as mentioned above. The said system is adapted for ex vivo or extra corporeal purification of blood which involves the removal of apoptotic chromatin fragments that are known to circulate in blood of normal persons and in higher quantities in patients with cancer and several other conditions with the help of a separating means.

It has been found that apoptotic chromatin fragments that circulate in blood exist in wide range of sizes measuring from ˜5 nm to ˜1200 nm. It has also been found that some of the physical properties of apoptotic chromatin fragments are similar to those of platelets. These two properties of apoptotic chromatin fragments allow for at least three different aspects for separation of apoptotic chromatin fragments from whole blood.

For the treatment according to present invention, the system may be capable of removing such apoptotic chromatin fragments from whole blood involving separation of plasma containing such apoptotic chromatin fragments from blood cells. The conventional process of separation of plasma from whole blood, called plasmapheresis, involves centrifugation using standard devices like Haemonetics® MCS® Haemonetics Corporation, USA or by passage of blood through conventional hollow fibre plasma filters that use filtration membranes with a pore size of ˜500 nm (e.g. Plasmaflux®, Fresenius AG, Germany). However, these devices cannot be employed for the purpose of the present invention since they either sediment the chromatin fragments together with the red bloods cells, white blood cells and platelets (centrifugation); or retain substantial amount of circulating chromatin within the hollow fibres (filtration). Therefore, for the purpose of the present invention specially designed plasma filters using principle of tangential filtration with membranes having appropriate pore size, more appropriately pore size of ˜1000-˜1500 nm are required. This generates a plasma fraction wherein most of, but not all, the apoptotic chromatin fragments are filtered out while RBCs, WBCs and platelets are retained. This chromatin rich plasma fraction is referred herein and throughout the description below as CRP (chromatin rich plasma).

Discarding CRP thus generated to remove the apoptotic chromatin cannot be employed, since this will result in loss of plasma which will require to be replaced by allogenic plasma with its inherent and potentially serious consequences. Further, this will lead to loss of other important constituents of plasma. Therefore, other means are involved for further processing of CRP to specifically clarify the plasma free of chromatin fragments which can be returned to the patient. Such means comprise chromatin removal chamber comprising means selected from i) contraption adapted for centrifugation at appropriate centrifugal force to sediment the chromatinfragments, and/or ii) immunological agents/antibodies (adsorbtion), and/or iii) chemical agents (adsorbtion), and iv) biochemical agents/enzymes (degradation).

The clarified plasma is reconstituted with the blood cells and re-infused back into the patient. The means adapted for reconstitution of blood after removal of apoptotic chromatin fragments comprise mixing chamber. Herein, reconstitution of blood free of apoptotic chromatin fragments is undertaken by mixing the clarified plasma derived from the separating means described above and the blood cells generated from filtration of whole blood described earlier at the time of generation of CRP.

In another aspect of the present invention the system for the treatment is capable of removing such apoptotic chromatin fragments from whole blood involving generation of a plasma fraction which includes all the chromatin fragments. However, in view of similarities in some of the physical properties of apoptotic chromatin fragments and platelets, this plasma fraction will also contain substantial amount of platelets. This desired plasma fraction that is rich in platelets and chromatin fragments will be referred herein as platelet and chromatin rich plasma (PCRP) throughout the description below. It may be noted that PCRP has higher amount of chromatin fragments than CRP. Hence the system according to this aspect comprises means for generation of PCRP by separating RBCs and WBCs from whole blood. The said means comprises sedimentation chamber involving passive sedimentation. Alternatively, the said means comprise filtration system having a membrane of appropriate porosity, more appropriately, pore size of ˜2000 nm-˜3000 nm, for tangential filtration. This will ensure a complete filtration of chromatin fragments but will also filter most of the platelets. The said means alternatively comprise contraption for sedimentation of RBCs and WBCs, but not platelets and chromatin fragments, by a centrifuge operating at an appropriate speed. The PCRP thus generated is passed through a CRP generation chamber which comprises a filter with membrane of pore size ˜1000-˜1500 nm as described above for generating CRP and retaining the platelets. Subsequently, the removal of apoptotic chromatin fragments from CRP is effected in a chromatin removal chamber. The said chromatin removal chamber comprises means selected from i) contraption adapted for centrifugation at appropriate centrifugal force to sediment the chromatin participles, and/or ii) immunological agents/antibodies (adsorbtion), and/or iii) chemical agents (adsorbtion), and/or iv) biochemical agents/enzymes (degradation). According to another aspect of the invention platelets are removed from PCRP with the help of a flowcytometry-assisted cell sorter leading to the generation of CRP. In the flowcytometry process PCRP is converted into a thin laminar stream in the flow chamber of this device. The platelets are segregated along the stream into a different path using system of lasers, photocells and electrostatic fields. The segregation can be done based on physical properties such as light scattering pattern, charge, fluorescence with labeled antibodies and such like. The CRP thus generated after removal of platelets from PCRP with the help of the flowcytometry-assisted cell sorter is treated for removal of apoptotic chromatin fragments. The removal of apoptotic chromatin fragments from CRP is effected in a chromatin removal chamber. The said chromatin removal chamber comprises means selected from i) contraption adapted for centrifugation at appropriate centrifugal force to sediment the chromatin participles, and/or ii) immunological agents/antibodies (adsorbtion), and/or iii) chemical agents (adsorbtion), and/or iv) biochemical agents/enzymes (degradation).

The means adapted for reconstitution of whole blood after removal of apoptotic chromatin fragments comprise mixing chamber. Herein, reconstitution of whole blood free of apoptotic chromatin fragments is undertaken by mixing i) the clarified plasma derived from the above separating means, ii) platelets generated from the filtration of PCRP in the CRP generation chamber, and iii) red and white blood cells generated in the PCRP generating chamber.

It has been found that the systems according to the above aspects of the present invention do not remove chromatin fragments from blood or plasma completely. In filtration of whole blood, according to first aspect involving means for generation of CRP, about 25% of the chromatin fragments are retained in blood while in the system according to the second aspect, involving means for filtration of PCRP, ˜15% are retained. It is, therefore, desirable to have additional means to improve the efficiency of the process further.

Hence according to another aspect, the system of the present invention is provided with means for successive removal of chromatin fragments by separating means comprising multiple chromatin removal chambers.

The system is provided with means for generating PCRP selected from those involving i) passive sedimentation, ii) tangential filtration using membranes of pore size ˜2000-˜3000 nm and iii) centrifugation of whole blood at an appropriate speed for sedimentation of RBCs and WBCs but not platelets and chromatin fragments. Thereafter, the system is provided with the first chromatin removal chamber as separating means adapted for treatment of PCRP. The said removal of apoptotic chromatin fragments from PCRP is then be achieved by means selected from i) immunological agents/antibodies (adsorption), and/or ii) chemical agents (adsorption), and/or iii) biochemical agents/enzymes (degradation), and/or iv) contraption adapted for density gradient centrifugation of PCRP to selectively precipitate chromatin fragments. It has been found that these means preferentially remove larger/denser chromatin fragments (˜500 nm-˜1200 nm) leaving behind smaller/lighter chromatin fragments (<˜500 nm) in plasma. In one aspect of the invention comprising multiple chromatin removal chambers, only the first chromatin removal chamber is employed wherein the above chromatin depleted platelet rich plasma can be reconstituted with RBCs and WBCs in a mixing chamber and returned to the subject. However, this will achieve only partial removal of apoptotic chromatin fragments.

Accordingly, in another aspect of the invention wherein more complete removal of the residual smaller (<˜500 nm) apoptotic chromatin fragments is achieved, the separating means comprise multiple chromatin removal chambers. In such a system the first chamber described above removes larger/denser apoptotic chromatin fragments. Subsequently, two more chromatin removal chambers are incorporated in the system for further removal of the smaller chromatin fragments. This second chromatin removal chamber comprises means for further removal of fine apoptotic chromatin fragments by filtration through membranes of appropriate porosity which will selectively retain the platelets and allow the fine chromatin fragments and plasma to be filtered out. For this purpose the commercially available plasma filtration device (Plasmaflux®, Fresenius AG, Germany), which uses hollow fibre membranes with a pore size of ˜500 nm, is used. This plasma filtrate that contains the finer chromatin fragments may be discarded thereby removing fine chromatin fragments from the body. However, this will also entail loss of useful plasma and its components. Reclaimation of plasma that is filtered out with fine chromatin fragments can be achieved by selectively removing these finer chromatin fragments in the third chromatin removal chamber of separating means of the system of present invention.

Accordingly, the system is provided with separating means having third chromatin removal chamber adapted to treatment of finer chromatin fragments (˜5 nm to ˜500 nm) in the platelet free plasma delivered in the filtrate from second chromatin removal chamber. The third chromatin removal chamber comprises means selected from (i) contraption adapted for centrifugation at appropriate centrifugal force to sediment the finer chromatin fragments, and/or ii) immunological agents/antibodies (adsorption), and/or iii) chemical agents (adsorption), and/or iv) biochemical agents/enzymes (degradation). The clarified plasma thus generated is led to the mixing chamber for reconstitution of blood.

The means adapted for reconstitution of blood after removal of apoptotic chromatin fragments comprises mixing chamber wherein reconstitution of whole blood free of apoptotic chromatin fragments is carried out. Reconstitution of whole blood is carried out by mixing i) clarified plasma from the third chromatin removal chamber, with ii) platelets from the second chromatin removal chamber and iii) red and white blood cells generated in the means adapted for the removal of PCRP from blood cells.

In the case of the system that employs only the first chromatin removal chamber reconstitution is carried out by mixing i) clarified plasma (chromatin depleted but not chromatin free) and platelets from the first chromatin removal chamber and ii) the red and white blood cells generated in the means adapted for the removal of PCRP from blood cells.

The means adapted to communicating and guiding of blood or plasma from the body to the said separating means through the means for removal of PCRP from blood cells, to means to reconstitute blood and direct the treated blood back into the body comprise conduits. Thus, blood enters the system of present invention via a conduit which communicates with a blood vessel of the subject via a suitable catheter. The conduit can comprise various types of flexible plastic tubings including, for example, non-thrombogenic materials such as heparinized polytetrafluroethylene (PTFE), heparinized surgical grade silicon rubber, medical grade polyvinylchloride (PVC) and the like. Blood may be drawn from the subject using a standard peristaltic pump. The amount and speed with which the blood is to be drawn may vary between 50-400 ml over a period of 1-10 minutes depending on the body habitus and physiological status of the subject. The conduit is provided with a suitable three-way valve that can be set to control the direction of flow of blood or fluid and interrupt the blood flow if needed. Further, to prevent clotting of blood in the extra-corporeal state an appropriate anticoagulant such as heparin will be added to blood at an appropriate dose ranging from 1-5 IU of heparin per ml of blood or a dose based on the body weight of the patient. The administration of anticoagulant can be effected from a reservoir using a standard commercially available infusion pump. It is also possible to add the anticoagulant in bolus doses by means of a syringe, the dose of the anticoagulant being based on the amount of blood being withdrawn for each cycle of processing by the device. The anticoagulated blood is led via an air trap to PCRP generation chamber. In a preferred embodiment this comprises of a sedimentation chamber for separation of PCRP from red and white blood cells. The blood is allowed to stand undisturbed in the sedimentation chamber for a period of 10-30 minutes. After passive sedimentation of red and white blood cells has been accomplished, the supernatant PCRP is drawn through an outflow conduit. The withdrawal of PCRP is effected by using a standard pump, which could be a peristaltic pump. The same pump also propels the blood to the first chromatin removal chamber.

PCRP hence drawn is led into the next chamber namely, the first chromatin removal chamber, that is described later. In one of the embodiments, the chromatin removal in the said chamber is effected by means selected from immunological, and/or chemical, and/or biochemical/enzymatic agents. The chamber contains matrices in the form microspheres, sheets or hollow fibre membranes etc. coated with appropriate antibodies, and/or chemical, and/or biochemical/enzymatic agents having high affinity for apoptotic chromatin fragments. The passage of PCRP through this chamber results in the selective removal of apoptotic chromatin fragments by affinity adsorption or degradation. An additional modification of this system is the incorporation of means that allow for online recharging/regeneration of the adsorption column. This comprises a reservoir that contains appropriate chemical agents like hypertonic saline and like that can be passed through the column when it is not in use with the help of a peristaltic pump. The regenerating solution can then be drained into a container before it is discarded. Since the above modification will interrupt the operations of the device, a further modification involves having two adsorption columns in parallel wherein one column could be in use for adsorption when the other is under regeneration cycle.

In another embodiment, the first chromatin removal chamber of the separating means could be a centrifugation device wherein the removal of chromatin is achieved by density gradient. PCRP is subjected to density gradient centrifugation with an appropriate medium and at an appropriate centrifugal force which selectively sediments the apoptotic chromatin fragments and retains the platelets and plasma in the supernatant.

The chromatin depleted platelet rich plasma obtained from the above two alternative embodiments of the first chromatin removal chamber is returned to the patient after reconstitution with red and white blood cells in a system comprising separation means with single chromatin removal chamber. However, it has been found that the above two processes do not completely remove all the chromatin fragments and hence the addition of further steps for the complete removal of chromatin fragments is desired. It has also been found that the chromatin that does not get removed comprises chromatin fragments which are smaller (<˜500 nm) than platelets. Since the chromatin depleted plasma obtained from the system comprising single chromatin removal chamber contains finer apoptotic chromatin fragments, the latter are removed from the said plasma by passing through a second chromatin removal chamber. This comprises a tangential filtration device comprising filtration membranes of appropriate porosity that retain the platelets and allow the finer chromatin fragments and plasma to be delivered in the filtrate. For this purpose the commercially available plasma filtration device (Plasmaflux®, Fresenius AG, Germany), which uses hollow fibre membranes with a pore size of ˜500 nm, is used.

At this juncture another preferred modification is the introduction of a recirculation loop for increasing the efficiency of chromatin removal. This involves recirculating the retentate from the hollow fibre filtration chamber (second chromatin removal chamber) through the first chromatin removal chamber followed by passage through the hollow fibre filtration chamber again. This is achieved by introduction of a three-way valve before the first chromatin removal chamber and one after the second chromatin removal chamber. The said valves can then be programmed to direct the flow through a conduit after the second chromatin removal chamber and reintroduce the retentate from the said chamber back into the first chromatin removal chamber. A peristaltic pump may drive the recirculation. The number of passages made in the recirculation loop will depend on cumulative maximum filtrate that is produced after the filtration chamber. The preferred cumulative filtration fraction is between 0.25 to 0.75 of the volume flown through. After the requisite number of recirculation cycles, the retentate from the filtration chamber (second chromatin removal chamber) that comprises platelets is led into the mixing chamber for reconstitution with red and white cells that is eventually reinfused to the subject.

The finer chromatin fragments present in the filtrate plasma from the second chromatin removal chamber are then separated in the third chromatin removal chamber which in its preferred embodiment is a centrifugation device. The said centrifugation device will subject the platelet free plasma fraction to an appropriate centrifugal force which will sediment the finer chromatin fragments. The supernatant containing clarified chromatin free plasma is then led into the mixing chamber with the help of a peristaltic pump for reconstitution with red and white blood cells and platelets for reinfusion to the subject. Thus, the mixing chamber receives inputs from the retentate fraction from the second chromatin removal chamber (containing platelets), chromatin free supernatant plasma from the third chromatin removal chamber and red and white cells from the PCRP generation chamber i.e. the sedimentation chamber. The reconstituted blood is then removed by a conduit and passed through a warmer to bring the blood to body temperature and then reinfused to the subject via an air trap to prevent air embolism. The movement to and from the mixing chamber is propelled by appropriate peristaltic pumps.

Another embodiment of the system for separation of chromatin from blood comprises means for generation of PCRP as described above. The PCRP thus generated is then direct to the first chromatin removal chamber. The said chamber being a flowcytometric cell sorter based device. Herein PCRP is converted into a thin laminar stream in the flow chamber of this device. The platelets are segregated along the stream into a different path using system of lasers, photocells and electrostatic fields. The segregation can be done based on physical properties such as light scattering pattern, charge, fluorescence with labeled antibodies and such like. The stream containing platelets is returned to the patient. The plasma fraction that has chromatin is then directed to another chromatin removal chamber similar to the third chromatin removal chamber which in its preferred embodiment is a centrifugation device as described above. The clarified chromatin free plasma from this chamber is then returned to the patient via the mixing chamber.

### DETAILED DESCRIPTION OF INDIVIDUAL SEPARATION CHAMBERS

The chamber for generation of chromatin rich plasma (CRP) to separate plasma fraction containing apoptotic chromatin fragments, from red and white blood cells and platelets is a plasma filter adapted for tangential filtration. According to the preferred embodiment this filtration device herein called CRP generation chamber comprises hollow fibres. The hollow fibres are made of membranes of appropriate porosity, more specifically, porosity of ˜1000-˜1500 nm which will retain the blood cells and allow most of the chromatin fragments to be filtered out together with plasma. The hollow fibres are placed in a housing with an inlet for entry of whole blood into the hollow fibres and an outlet for outflow of retentate with blood cells. The space around the hollow fibres in the housing is the filtrate chamber wherein CRP is collected. The CRP thus obtained is removed from a collection port provided in the filtrate chamber. The housing material is made of medical grade sterilizable synthetic polymers (e.g. polypropylene, polysulfone, polystyrene, polycarbonate, etc.) and the like. The housing can have a volume 100-300 ml with a length/diameter ratio between 2:1 to 5:1. The hollow fibre membrane is selectively made of polymers such as polysulphone, poly-acrylo nitirile, polypropylene, polycarbonate, polyethersulphone and the like as used in dialyzers and conventional plasmafilters. The priming volume of the hollow fibres may range from 20-100 ml. The positive pressure inside the hollow fibres generated from the peristaltic pump leads to the production of the filtrate (CRP) from whole blood. Additionally, a pump is connected to the collection port to create a negative pressure inside the filtrate chamber to facilitate this process. The total transmembrane pressure is kept at its minimum, preferably below 100 mm Hg, to prevent haemolysis of the blood traversing the filter device.

In another embodiment, the means for separation of CRP from blood cells comprises a plasma filter adapted for tangential filtration wherein the filtration membrane of similar material and pore size as described above is used. The membranes can be in the form of plain or pleated sheet(s) that could be stacked up in flat configuration or be placed in the form of coils. The membranes are placed in a housing similar to that described for the hollow fibre filtration device.

The means for separating apoptotic chromatin fragments from CRP generated from either of the above filtration devices comprises a centrifugation or an adsorption device. In a preferred aspect, CRP is directed to a suitable chamber wherein centrifugation is carried out in a standard centrifugation machine with appropriate rotor to sediment the chromatin fragments. The centrifugation is carried out in one or more containers of 25-300 ml volume each, with length to diameter ratio of 2:1 to 5:1. The containers are transparent and made of medical grade sterilizable synthetic polymers (e.g. polypropylene, polystyrene, polycarbonate, etc.) and the like. Each container will have an inlet and an outlet connected to suitable conduits. The containers should be strong enough to withstand centrifugation at 20,000-30,000×g. The centrifugation is carried out for 2-20 minutes. The supernatant, which is chromatin free plasma, is carefully delivered via the outlet conduit. It has been found that high speed centrifugation of the plasma fraction results in 95-97% sedimentation of chromatin fragments and thus clarifying the plasma fraction which is returned to the subject.

In another aspect, the means for separating apoptotic chromatin fragments from CRP comprise an adsorbtion device selected for removal of apoptotic chromatin fragments selected from matrices coated with appropriate agents selected from immunological agents (such as antibodies with affinity for chromatin) or chemical agents (such as DEAE or such like cationic resins) or biochemical/enzymatic agents (such as DNA degrading enzymes). Passage of CRP through such matrices leads to the removal by adsorption/binding/degradation of chromatin fragments contained in CRP. The matrices may be in the form of sheets or membranes with a large surface area, hollow fibres, beads or fibrous wool. These could be made of natural/synthetic polymers like cellulose, modified cellulose, polycarbonate, polysulphone, polystyrene, polyether suphone, or such like, glass, ceramics and such like. These matrices are coated with the agents (immunological such as antibodies with affinity for chromatin or chemicals such as DEAE or such like cationic resins) or biochemical/enzymatic agents (such as DNA degrading enzymes). The housing for these coated matrices has an inlet and an outlet with a priming volume varying between 30-300 ml. The amount of agent coated will depend on the activity of the agent, the expected volume of plasma to be purified and duration of the process. In one preferred aspect 0.5-20 mg of antibody will be coated for clarifying/processing 50-500 ml of CRP. The antibody could be one or more of anti histone H1, H2A, H2B, H3, H4, anti-DNA either used alone or in combinations thereof. It has been found that the proportion of apoptotic chromatin removed from CRP can be up to 90% depending on exposure time, amount of antibody, affinity of antibody etc. It has been seen that incremental chromatin adsorption takes place from the time CRP comes in contact with the adsorption ligands up to 2 hrs of exposure.

The means for reconstitution of whole blood free of apoptotic chromatin fragments comprises mixing chamber adapted for mixing clarified plasma generated after centrifugation or adsorbtion of CRP with red and white blood cells and platelets generated by filtration of whole blood in the CRP generation chamber. The preferred embodiment of the mixing chamber is a cylindrical chamber made of medical grade sterilizable synthetic polymers (e.g. polypropylene, polysulfone, polystyrene, polycarbonate, etc.) and the like. The cylinder can have a volume of 50-500 ml with a length/diameter ratio between 2:1 to 5:1. The housing has two inlets and an outlet. The first inlet delivers red and white blood cells and platelets from the CRP generation chamber and the second inlet receives the clarified plasma from the chromatin removal chamber. The mixing of the above components of blood is achieved by mechanical movements. The reconstituted blood is removed by a conduit through the outlet.

In another embodiment of the mixing chamber the housing is made of flexible plastic like plasticised polyvinylchloride (PVC) and the like with two inlets and an outlet with appropriate conduits attached. The volumes and length:diameter ratio being similar to the mixing chamber described above. The flexible nature of the housing allows delivery of the reconstituted blood by graduated extrinsic compression.

In the process utilizing generation of PCRP, the means for separating PCRP from red and white cells is PCRP generation chamber which comprises means selected from filtration chamber, centrifugation chamber and a sedimentation chamber. The sedimentation chamber is a cylindrical container made of medical grade sterilizable synthetic polymers (e.g. polypropylene, polysulfone, polystyrene, polycarbonate, etc.) and the like. The cylinder can have a volume of 50-500 ml with a length/diameter ratio between 2:1 to 5:1. The housing has an inlet and an outlet each having a sampling/injection port for collecting samples or adding additives like anticoagulant. The inlet delivers the blood from the subject. After the process of passive sedimentation for 10-30 minutes the supernatant PCRP is removed by a conduit through the outlet. The conduit is so designed that lower end of the conduit can be adjusted to position it within the chamber just above the upper level of sedimented red and white cells. Further, the chamber is provided with a drain at the bottom with appropriate conduit and a valve to deliver the sedimented red and white blood cells to the mixing chamber for reconstitution of blood with clarified plasma and platelets at the end of the process.

In another embodiment of the sedimentation chamber, the housing is made of flexible plastic like plasticised polyvinylchloride (PVC) and the like with an inlet, outlet and drain port with appropriate conduits attached. The volumes and length to diameter ratio being similar to the sedimentation chamber described above. The flexible nature of the housing allows delivery of the supernatant PCRP by graduated extrinsic compression and the same could be done for delivery of sedimented red and white blood cells for reconstitution of blood at the end of the process.

In another embodiment, the separation of PCRP from red and white cells is carried out by means of a specially designed plasmafilter adapted for tangential filtration. This specialized plasmafilter comprises porous membranes in the form of hollow fibres placed in a housing with an inlet for entry of whole blood into the hollow fibres and an outlet for outflow of retentate with blood cells. The space around the hollow fibres in the housing is the filtrate chamber. The membrane is specifically designed such that the pore size is between ˜2000-˜3000 nm to retain the red and white blood cells and at the same time to allow the PCRP to filter out in the filtrate chamber of the housing. The filtrate thus obtained is removed from a collection port provided in the filtrate chamber. The housing material is made of medical grade sterilizable synthetic polymers (e.g. polypropylene, polysulfone, polystyrene, polycarbonate, etc.) and the like. The housing can have can have a volume 100-300 ml with a length/diameter ratio between 2:1 to 5:1. The hollow fibre membrane could be made of polymers such as polysulphone, poly acrylo nitirile, polypropylene, polycarbonate, polyethersulphone and the like as used in dialyzers and conventional plasmafilters. The priming volume of the hollow fibres ranges from 20-100 ml. The positive pressure inside the hollow fibres generated from the peristaltic pump leads to the production of the filtrate from whole blood. Additionally, a pump is connected to the collection port to create a negative pressure inside the filtrate chamber to facilitate this process. The total transmembrane pressure needs to be kept at its minimum, preferably below 100 mm Hg, to prevent hemolysis of the blood traversing the filter device.

In another embodiment, the separation of PCRP from red and white cells is carried out by means of a specially designed plasmafilter adapted for tangential filtration wherein the filtration membrane of similar material and pore size as described above is housed in standard filtration housing and is in the form of plain or pleated sheet(s) that could be stacked up in flat configuration or be placed in the form of coils.

In another embodiment, the means for generation of PCRP from red and white blood cells comprises a centrifugation chamber with appropriate rotor to sediment red and white cells but not the chromatin fragments and platelets. The centrifugation is carried out at an appropriate speed in one or more containers of 25-300 ml volume each, with length to diameter ratio of 2:1 to 5:1. The containers will be transparent and made of medical grade sterilizable synthetic polymers (e.g. polypropylene, polystyrene, polycarbonate, etc.) and the like. Each container will have an inlet and an outlet connected to suitable conduits. The centrifugation is carried out for 2-20 minutes. The supernatant, which is PCRP, is carefully delivered via the outlet conduit.

The device for separating apoptotic chromatin fragments from PCRP could be selected from several embodiments. One of the embodiments comprises device adapted for tangential filtration. This filter has a membrane porosity of ˜1000-˜1500 nm and is identical to the CRP generation chamber already described earlier for the filtration of whole blood to separate plasma fraction containing chromatin fragments (CRP). The means for separating apoptotic chromatin fragments from the filtered plasma fraction comprises a centrifugation or an adsorption device already described above in context of clarification of CRP generated from whole blood filtration. In another embodiment for the separation of chromatin fragments from platelets, PCRP is passed through a device based on the principles of flowcytometry-assisted cell sorter or similar processes. In the flowcytometric process PCRP is converted into a thin laminar stream in the flow chamber of this device. The platelets are segregated along the stream into a different path using system of lasers, photocells and electrostatic fields. The segregation can be done based on physical properties such as light scattering pattern, charge, fluorescence with labeled antibodies and such like. The plasma fraction containing apoptotic chromatin fragments (CRP), but not platelets, is led into a chromatin removal chamber. The said chromatin removal chamber comprises a centrifugation or an adsorption/degradation device already described above in context of clarification of CRP generated from whole blood filtration.

Further refinement of the process of removal of apoptotic chromatin fragments from PCRP comprises a system involving multiple chromatin removal chambers. Such chambers are described in another aspect.

The separating means comprising the first chromatin removal chamber for removal of apoptotic chromatin fragments from PCRP comprises matrices coated with appropriate agents selected from immunological agents (such as antibodies with affinity for chromatin) or chemical agents (such as DEAE or such like cationic resins) or biochemical agents/enzymes (such as DNA degrading enzymes). Passage of PCRP through such matrices leads to the removal by adsorption/binding/degradation of the chromatin fragments contained in PCRP. The matrices may be in the form of sheets or membranes with a large surface area, hollow fibres, beads or fibrous wool. These could be made of natural or synthetic polymers like cellulose, modified cellulose, polycarbonate, polysulphone, polystyrene, polyether suphone, or such like, glass, ceramics and such like. These matrices are coated with agents such as immunological (antibodies with affinity for chromatin) or chemicals (such as DEAE or such like cationic resins) or biochemical (such as DNA degrading enzymes). The housing for these coated matrices has an inlet and an outlet with a priming volume varying between 30-300 ml. The amount of agent coated will depend on the activity of the agent, the expected volume of PCRP to be purified and duration of the process. In one preferred aspect 0.5-20 mg of antibody will be coated for clarifying/processing 50-500 ml of PCRP. The antibody could be one or more of anti histone H1, H2A, H2B, H3, H4, anti-DNA either used alone or in combinations thereof. It has been found that the proportion of apoptotic chromatin fragments removed from PCRP can be up to 90% depending on exposure time, amount of antibody, affinity of antibody etc. It has been seen that incremental chromatin adsorption takes place from the time PCRP comes in contact with the adsorption ligands for up to 2 hrs of exposure. This is not associated with any significant loss of platelets.

In another preferred embodiment of the first chromatin removal chamber, PCRP generated is led to a suitable chamber wherein density gradient centrifugation is carried out to selectively sediment chromatin fragments. Density gradient centrifugation is carried out in one or more containers of 50-300 ml volume each, with length to diameter ratio of 2:1 to 5:1. The containers are transparent and made of medical grade sterilizable synthetic polymers (e.g. polypropylene, polysulfone, polystyrene, polycarbonate, etc.) and the like. Each container has an inlet, an outlet and a drain port at the bottom with a valve. The containers should be strong enough to withstand centrifugation at 200-1000×g. The inlet and outlet are connected to suitable conduits. The density gradient for centrifugation is created by using a suitable medium like mixtures of polysaccharide and radio-opaque contrast medium like Histopaque-1077® (Solution of polysucrose and sodium diatrizoate adjusted to density of 1.077 +/− 0.00 lg/ml; Sigma Diagnostics®) and such like. This medium is used in 1:1 ratio with the PCRP and centrifuged for 2-20 minutes. The larger/denser chromatin fragments sediment in the density gradient medium with a minimal loss of platelets. The supernatant, which is chromatin depleted platelet rich plasma, is carefully delivered via the outlet conduit. The lower end of this outlet conduit is adjustable to a position just above the density gradient medium. The drain at the bottom can then be used to reject the used up density gradient medium. It has been found that 30%-60% of the chromatin fragments are selectively sedimented from PCRP into the density gradient medium. The effective loss of platelets during this process is <15%.

The second chromatin removal chamber involves further clarification of chromatin depleted platelet rich plasma generated in first chromatin removal chamber that still has some residual fine chromatin fragments. This can be achieved by filtration through membranes of appropriate porosity, ˜100-˜1000 nm preferably ˜500 nm, which will selectively retain the platelets and allow the fine chromatin fragments to be filtered out. The device for carrying out this filtration could be similar to commercially available plasmafilters like Plasmaflux®, Fresenius AG, Germany and such like. It has been found that the passage of chromatin depleted platelet rich plasma with residual finer chromatin fragments through this filter leads to 75-100% filtration of finer chromatin fragments with complete retention of platelets which are ultimately returned to the subject.

The third chromatin removal chamber comprises means adapted to remove finer chromatin fragements from the filtered plasma fraction that is generated in the second chromatin removal chamber. In a preferred aspect, the said filtrate from second chromatin removal chamber is directed to a suitable chamber wherein centrifugation is carried out in a standard centrifugation device with appropriate rotor to sediment fine chromatin fragments. The centrifugation is carried out in one or more containers of 25-300 ml volume each, with length to diameter ratio of 2:1 to 5:1. The containers will be transparent and made of medical grade sterilizable synthetic polymers (e.g. polypropylene, polystyrene, polycarbonate, etc.) and the like. Each container has an inlet and an outlet connected to suitable conduits. The containers should be strong enough to withstand centrifugation at 20,000-30,000×g. The centrifugation is carried out for 2-20 minutes. The supernatant, which is chromatin free plasma, is carefully delivered via the outlet conduit. It has been found that high speed centrifugation of platelet free plasma that has fine chromatin fragments as described above results in 95-97% sedimentation of these particles thus clarifying the plasma fraction which is returned to the subject.

In another embodiment of the third chromatin removal chamber, the separation of finer chromatin fragments from plasma free of platelets generated in second chromatin removal chamber is achieved by directing this plasma through a chamber similar to first chromatin removal chamber. Herein the separating means for removal of apoptotic chromatin fragments comprise matrices coated with appropriate agents selected from immunological agents (such as antibodies) or chemical agents (such as DEAE or such like, cationic resins) or biochemical agents (such as DNA degrading enzymes). Passage of plasma through such matrices leads to further removal by adsorption/binding/degradation of the finer chromatin fragments further clarifying plasma.

The means for reconstitution of blood comprises mixing chamber. The mixing chamber is similar to the one described above for the first method and is adapted for reconstituting whole blood free of apoptotic chromatin fragments by mixing i) the clarified plasma from third chromatin removal chamber with ii) the platelets generated in second chromatin removal chamber with iii) red and white blood cells generated in the PCRP generation chamber.

The conduits used in the whole system comprise various types of standard flexible plastic tubings including, for example, non-thrombogenic materials such as heparinized polytetrafluroethylene (PTFE), heparinized surgical grade silicon rubber, medical grade polyvinylchloride (PVC) and the like. The size range of these conduits is 2-10 mm internal diameter as appropriate.

Plasma chromatin levels for the development of the device are measured by using the Cell Death Detection Elisa®, supplied by Roche Diagnostics GmBH. The assay is based on quantitative sandwich-enzyme immunoassay principle using two different mouse monoclonal antibodies directed against DNA and histones. It involves fixation of anti-histone antibody by adsorption on the wall of the microplate module coated with streptavidin. This is followed by binding of nucleosomes (chromatin fragments) contained in the sample via their histone components to the immobilized anti-histone antibody. Subsequently, the anti-DNA monoclonal antibody conjugated with peroxidase binds to the DNA component of the nucleosome and the amount of peroxidase retained in the immunocomplex reacts with 2,2′-azino-bis-(3-ethylbenzthiazoline-6-sulfonate) (ABTS) as a substrate to produce a coloured reaction as a measure of the amount of nucleosomes present in the sample.

Accordingly by the system of the present invention removal of apoptotic chromatin fragments in circulation is achieved for preventing DNA damage leading to genomic instability, senescence, apoptosis and oncogenic transformation within the body as a method of treatment/prevention of associated pathological conditions Chromatin fragments are known to circulate in blood of normal persons and in higher quantities in several pathological conditions including cancer. Since the present inventors have found that such chromatin fragments are capable of inducing DNA damage leading to genomic instability, senescence, apoptosis and oncogenic transformation in healthy cells on being ingested by them, and since they may also be released from existing tumors, especially following chemo- radiotherapy which may lead to further spread of cancer in the body (metastasis), their removal would prevent initiation and spread of cancer in the body andretard/prevent many other pathological processes. The present invention clearly shows that such potentially harmful apoptotic chromatin fragments can be removed by ex-vivo or by extra corporeal purification of blood using the said system.

Since that the present inventors have found that circulating chromatin fragments are capable of inducing progressive DNA damage leading to genomic instability, senescence, apoptosis and oncogenic transformation in normal cells, it is obvious that this process also contributes to the pathological processes akin to ageing and age related degenerative diseases such as neurodegenerative diseases, atherovascular diseases, diabetes etc. For example, it is already known that genomic instability, senescence and apoptosis of cells is associated with increasing age of an individual. The removal of apoptotic chromatin fragments by ex-vivo or extra corporeal purification of blood could, therefore, prevent or retard the progression of ageing and age related conditions.

Since many other conditions, such as inflammation, infections, sepsis and multi-organ system failure, renal failure and autoimmune diseases, are associated with increased apoptosis, removal of apoptotic chromatin fragments by ex-vivo or extra corporeal purification of blood may prevent or ameliorate these conditions or their secondary pathological consequences.

Since viruses are capable of spreading from one cell to another through transfer of apoptotic bodies from virus infected cells, ex-vivo or extra corporeal removal of such virus infected apoptotic bodies/chromatin fragments could prevent spread of viral infections, such as HIV, within the body.

Since blood and blood products are used extensively in various medical situations, removal of apoptotic chromatin fragments by ex-vivo or extra corporeal purification of blood or blood products may prevent the harmful effects on the recipient originating from this exogenous burden of chromatin.

The separating means which can effectively remove apoptotic chromatin fragments ex-vivo is thus fabricated to purify blood and thereby eliminate fragments of apoptotic chromatin but not any other component of blood as a method of treating the associated disease conditions.

The invention is now described by way of illustrative, non-limiting examples and illustrations.

### EXAMPLES

Cell Culture:

It is sought to be demonstrated that when apoptotic chromatin fragments derived from normal or cancerous cells or those purified from serum/plasma of normal subjects and patients suffering from several disease conditions including cancer are added to recipient cells in culture, the chromatin fragments are ingested by them wherein they get integrated in their genomes and induce DNA damage, chromosomal instability, senescence, apoptosis, oncogenic transformation and other deleterious effects. The various recipient and donor cells used for the purpose are listed below. All cell lines are obtained from American Type Culture Collection (ATCC), USA. The ATCC Numbers are as follows:
NIH3T3 (ATCC No.: CRL-1658)—Embryonic mouse fibroblast
B16F10 (ATCC No.: CRL-6475)—Metastatic mouse melanoma
Jurkat (ATCC No.: CRL-TIB-152)—Human lymphocytic leukemia
NCTC Clone 1469 (ATCC No.: CCL-9.1)—Normal mouse liver
MM55.K (ATCC No.: CRL-6436)—Normal mouse kidney
B/CMBA.Ov (ATCC No.: CRL-6331)—Normal mouse ovary
MRC-5 (ATCC No. CCL-171)—Human lung fibroblast
HCN-1A (ATCC No. CRL-10442)—Human cortical neuron
   The NIH3T3 cells (ATCC No. CRL-1658) are cloned, and those clones which do not form colonies in soft agar are also used for some of our experiments. All cells except Jurkat, NCTC clone 1469 mouse liver and MRC-5 human fibroblast are grown in Dulbecco's Modified Eagle's Medium (DMEM) containing 10% fetal calf serum (FCS). Jurkat cells are grown in Roswell Park Memorial Institute (RPMI) medium supplemented with 10% FCS. NCTC Clone 1469 mouse liver cells are grown in DMEM containing 10% horse serum while MRC-5 human fibroblast cells are grown in Modified Eagle's Medium (MEM) containing 10% FCS.
   Induction of Apoptosis:

B16F10 and NIH3T3 cells are grown to a density of 10⁶ cells per 100 mm petri dish and are treated with Adriamycin (5 μg/ml). More than 95% of the cells undergo apoptosis as assessed by flowcytometry after 48 hours of treatment with respect to B16F10 cells and 5 days with respect to NIH3T3 cells. The cells are centrifuged at 600×g for 5 minutes and the pellets (P1) are washed 5 times with phosphate buffered saline (PBS) and the final pellets are suspended in 500 μl of complete culture medium. Jurkat cells are grown in 25 cm² flasks to a density of 10⁶ cells per ml and apoptosis is induced for 48 hours with 0.5 μg/ml of anti-Fas mAb (Roche Biochemicals). The apoptotic cells (>95%) are washed ×3 with PBS and the final pellet (P1) is suspended in 500 μl of complete culture medium.

The supernatant (S1), obtained after removal of the above (P1) apoptotic cells/bodies, also retains transforming activity. This activity is traced to apoptotic particles present in S1 fraction by further fractionation. S1 is centrifuged successively at 27,500×g for 20 minutes and 105,950×g for 40 minutes to generate pellets P2, P3 and supernatants S2, S3 respectively. S3 is centrifuged further at 346,410×g for 16 hours to yield pellet P4 made up of the smallest apoptotic particles that are detected to be active in the assay system.

Purification of Apoptotic Chromatin Fragments from Plasma/Serum:

0.5 ml of streptavidin coated sepharose beads are packed on to a polystyrene column above glass wool. The column is equilibrated with 2 ml of PBS. Biotinylated antihistone antibodies are added to the column and allowed to enter the gel bed. The bottom and top caps are sequentially replaced and incubated for 2 hours at room temperature. Following incubation the column is washed with PBS and 1 ml of plasma/serum is applied to the column and incubated for 2 hours. The plasma/serum is allowed to flow through, the column washed with PBS, and the apoptotic chromatin fragments bound to the biotinylated antibodies is immuno-eluted using 1 ml of 1M 0.9% NaCl solution. The eluate containing immunopurified apoptotic chromatin fragments is ultracentrifuged at 346,410×g for 16 hours to yield a pellet which is resuspended in PBS until use.

Plasma/serum are obtained from patients with various diseases such as diabetes, renal failure and sepsis as well as from age and sex matched controls. For cancer patients, samples are obtained 24-48 hours after the first course of chemo- or radiotherapy. Plasma/serum are separated by allowing the blood samples to stand at room temperature for 2 hours and collecting the supernatant plasma/serum fractions.

Levels of Apoptotic Chromatin Fragments in Plasma/Serum are Increased in Several Human Disease Conditions:

Chromatin concentration in plasma/serum is measured using a quantitative sandwich enzyme immunoassay which uses mouse monoclonal antibodies directed against both DNA and histones (Cell Death Detection ELISA Plus, Roche AS and MD, Germany). It is observed that the level of chromatin is increased in various diseases such as diabetes, renal failure, sepsis and cancer when compared to healthy subjects. In cancer patients a further increase in chromatin level is seen after chemo- or radiotherapy.

Nature of Apoptotic Particles:

DNA (with ³H-Thymidine) and proteins (with ³⁵S-Methionine) of chromatin of donor cells are metabolically labeled in separate experiments, induced to undergo apoptosis, and then both types of labeled cells and their breakdown products are used for size fractionation as described above. For this, semi-confluent B16F10 cells are metabolically pre-labeled with either ³H-Thymidine (5 μCi/ml) or ³⁵S-Methionine (100 μCi/ml) for 48 and 24 hours respectively. The cells are rendered apoptotic with adriamycin and the chromatin fragments are size fractionated by differential centrifugation. The pellets (P1-P4) are processed for electron microscopy (EM) using standard procedures. Briefly, the pellets are fixed in 3% glutaraldehyde in 0.1 M cacodylate buffer (pH 7.4) for 1 hour at 40° C. and post-fixed in 1% OsO₄ for 1 hour at 40° C. They are dehydrated in graded alcohol, embedded in araldite mixture and incubated for 48-72 hours at 60° C. for polymerization. Ultra-thin sections (600-800 Å) of the blocks are cut using glass knives on LKB 2088 Ultratome® V and mounted on double coated (formvar and carbon) 200 mesh copper grids (Pelco, USA). Grids are coated for autoradiography using EM 1 emulsion (Amersham), exposed for varying periods and developed with D19 developer [Fakan, S. and Fakan, J. Autoradiography of spread molecular complexes. In Electron Microscopy in Molecular Biology: a practical approach. Sommerville, J. and Scheer, U., (eds) IRL Press, Oxford, p. 201-214, 1987]. The sections are counterstained with a mixture of uranyl acetate and lead citrate and examined under Zeiss EM 109 electron microscope operating at 80 kV mode.

Under EM, the chromatin fragments are found to have the following average dimensions (N=20): Pellet 1: 346±164 nm (Mean±SD); Pellet 2: 161±53 nm; Pellet 3: 25±6 nm; Pellet 4: 8±2 nm. The particles in pellets 1 and 2 are relatively large and present a convoluted appearance on EM, hence their true size may be underestimated. It is possible that chromatin fragments even smaller than 8±2 nm are also present but they are not pursued in the present example.

Autoradiography and electron microscopy (EM) reveal that the fractionated radioactively labeled pellets consist of discrete particles containing both DNA and protein. This is clear evidence that the apoptotic particles are nothing other than fragments of chromatin.

Apoptotic chromatin fragments that are purified from plasma/serum are examined by electron microscopy using phosphotungstic acid negative staining procedure. The chromatin fragments having characteristic beaded appearance are clearly seen and they vary in size from a few nanometers to 1200 nanometers. However, the particles are often convoluted and hence their true sizes cannot be accurately ascertained.

Treatment of Recipient Cells with Apoptotic Chromatin Fragments Leads to their Internalization:

NIH3T3 cells are treated with apoptotic pellet P1 in a proportion of 1:1. Recipient NIH3T3 cells are grown to a density of 2×10⁵ cells per 60 mm petri dish and the apoptotic pellets suspended in 500 μl of culture medium are added directly to the recipient cells.

For EM-autoradiography, NIH3T3 cells are treated with labeled chromatin fragments from apoptotic cells. The recipient cells are washed and harvested on day 2 or 3 by scraping, centrifuged, and the pellets fixed and processed for EM-autoradiography as described above.

EM-autoradiography of sections of such NIH3T3 cells that are treated individually 48-72 hours earlier with apoptotic pellet P1 labeled either with ³H-Thymidine or with ³⁵S-Methionine reveal that both types of labeled particles of similar physical characteristics are present within the recipient cells both in the cytoplasm and in the nucleus. This indicates that the apoptotic fragments are rapidly ingested by the recipient NIH3T3 cells and enter their nuclei. Particles finer than P1 are visible within the cells/nuclei which suggests that P1 might be undergoing further intracellular processing/degradation.

Internalization of apoptotic chromatin fragments that are purified from serum into human lymphocytes is investigated by labeling the apoptotic chromatin fragments by the TUNEL method using Alexa labeled 5′-dUTP and examining the cells under fluorescent microscope. The presence of labeled particles are clearly visualized inside the lymphocytes indicating that the apoptotic chromatin fragments are internalized.

Ingested Apoptotic Chromatin Fragments are Incorporated into Recipient Cell Genomes:

The fact that the ingested apoptotic chromatin fragments are incorporated into the genome of recipient cells is demonstrated by fluorescence in situ hybridization (FISH). FISH protocol is followed essentially as per the original method of Pinkel, et al. [Pinkel, D., Straume, T. & Gray, J. W. Cytogenetic analysis using quantitative, high sensitivity, fluorescence hybridization. Proc. Natl. Acad. Sci. USA 83, 2934-2938, (1986)]. Metaphase spreads are prepared after colcemid treatment and the slides are examined under fluorescence microscope fitted with a cooled CCD camera. The human whole genomic and human pan-centromeric probes are obtained from CHROMBIOS GmbH. Mouse pan-centromeric probes are also obtained from the same source.

NIH3T3 cells which are treated with P1 (Jurkat) for 48 hours are hybridized with human whole genomic and human pan-centromeric painting probes. The presence of fragments of human genomic DNA as well as human centromeres in NIH3T3 mouse fibroblast cells is clearly revealed by FISH both in interphase and metaphase preparations.

NIH3T3 cells that are treated 6-48 hours earlier with apoptotic chromatin fragments purified from serum/plasma from healthy individuals and patients with cancer pre-treated 24-48 hrs earlier with chemo- or radiotherapy are similarly examined by FISH. The presence of fragments of human genomic DNA as well as human centromeres in NIH3T3 mouse fibroblast cells is clearly revealed by FISH both in interphase and metaphase preparations.

The above FISH experiment provide unambiguous evidence that apoptotic chromatin fragments derived from both cultured apoptotic cells as well as serum of healthy individuals and patients with cancer are not only ingested by recipient cells but that they are also incorporated in their genomes.

Ingested Apoptotic Chromatin Fragments Cause DNA Damage:

Apoptotic chromatin fragments purified from serum/plasma from healthy individuals and patients with cancer pre-treated 24-48 hrs earlier with chemo- or radiotherapy are added to various recipient cells. The recipient cells are fixed in 4% formaldehyde for 1 hour and immuno-stained with antibody to γH2AX—phosphorylated at ¹³⁹serine residue. The cells are examined by fluorescent microscopy. Signals indicating DNA damage can be clearly detected as early as 6 hours reaching a maximum at 24 hours when apoptotic chromatin fragments purified from as little as 20 μl of serum from pre-treated cancer patients is used. However, in case of healthy subjects, these changes are only observed when apoptotic chromatin fragments purified from serum volume greater than 100 μl are used.

Ingested Apoptotic Chromatin Fragments Induce Chromosomal/Genomic Instability:

DNA damage induced by apoptotic chromatin fragments leads to severe chromosomal instability in the recipient cells. Following treatment with apoptotic chromatin fragments, chromosomal changes in recipient cells are detectable as early as 24-48 hours. Various recipient cells are treated for 2-3 days either with apoptotic P1 pellets from B16F10 or Jurkat cells or apoptotic chromatin fragments purified from plasma/serum from healthy individuals and patients who had been treated for cancer. The recipient cells are arrested in metaphase with 0.03 μg/ml of colcemid for several hours are used. Air-dried chromosome preparations are prepared and at least 50 Giemsa stained metaphases from each study are scored for documentation of chromosomal abnormalities/rearrangements.

As much as 70%-80% of the metaphases examined show a wide range of non-specific and mitotically unstable chromosomal aberrations when apoptotic P1 pellet derived from B16F10 or Jurkat are used. These include multiple chromosomal and chromatid breaks and deletions; translocations involving multiple chromosomes; chromosomal fusions; ring chromosomes; di- and tricentric chromosomes; telomeric associations; amplifications—both centromeric and non-centromeric; centromeric elongation; double minutes and chromatid appositions. Similar changes are seen when apoptotic chromatin fragments purified from 10-20 μl of serum collected from patients with cancer after 24-48 hrs of chemo- or radiotherapy are used. However, in case of healthy subjects, these changes are only seen when apoptotic chromatin fragments purified from serum volume greater than 500 μl are used.

The extent of chromosomal instability is further highlighted when FISH experiments are done using a mouse pan-centromeric probe. Large scale and unusual centromeric amplifications are seen in the recipient NIH3T3 cells treated 48 hours earlier with P1 from apoptotic B16F10 cells.

For detection of genomic instability, a primer (CA)₈ anchored with 5′-RG, (where R is an equimolar mixture of adenosine and guanosine) is used for amplification of genomic DNA between the regions of CA repeats, as per the method of Stoler, et al.[Stoler, D. L. et al. The onset and extent of genomic instability in sporadic colorectal tumor progression. Proc. Natl. Acad. Sci. 96, 15121-15126 (1999)]. Treatment of recipient cells by apoptotic chromatin fragments purified from sera of post-treatment cancer patients produces genomic instability as indicated by several deletions and amplifications of the recipient cell DNA bands when PCR fragments are separated by PAGE and visualized by autoradiography. These changes are clearly visible with respect to band sizes between 200-900 bp.

Ingested Apoptotic Chromatin Fragments Induce Aneuploidy in Recipient Cells:

Flowcytometry is used to assess the temporal changes in the genomic DNA content of recipient cells after apoptotic chromatin treatment. When apoptotic B16F10 pellet P1 is used as chromatin donors and NIH3T3 cells as recipients, the earliest discernible effect is an increase in the S-phase fraction seen as early as 6 hours post treatment. This is followed by a G2/M block, clearly seen at 12 hours that gradually increases until a maximum is reached at 24 hours when 74% of the cells are arrested in this phase. The cells are apparently aneuploid by 48 hours, a condition which progressively becomes more pronounced reaching 95% at the end of 120 hours. The extent of genomic instability is apparently so severe that a significant fraction of recipients are unable to sustain a functional genome and undergo increasing apoptosis with passage of time. Similar changes are seen when apoptotic chromatin fragments purified from 100 μl of serum from cancer patients who had been treated with chemo-radiotherapy are used. The apoptotic chromatin fragments purified from healthy subjects are far less effective in producing the above changes.

Flowcytometry is performed using a FACS Calibur machine (Becton Dickinson, Mountain View, Calif.). For DNA analysis, cells are removed at various time points, fixed in 70% ethanol, stained with propidium iodide (50 μg/ml) and FL2 (A) is measured using 488 nm excitation and emission through >600 nm band pass filter on linear scale.

Apoptotic Chromatin Fragments Induce Senescence in Recipient Cells:

Induction of senescence is assessed on the basis of: 1) cellular morphology; 2) persistence of DNA damage by immunodetection of phosphorylated γH2AX; 3) Up-regulation of p53. Treatment of recipient NIH3T3 cells with apoptotic chromatin fragments purified from as little as 25 μl of serum from patients with cancer pre-treated with chemo- or radiotherapy induces a senescent phenotype in the recipient cells after a single application within 4-5 days. On the other hand, in case of apoptotic chromatin fragments purified from healthy subjects, as much as 1000 μl of serum failed to induce a senescent state on single application. However, upon repeated applications on every alternate day on 5-6 occasions, apoptotic chromatin fragments purified from only 100 μl of serum from healthy subjects was able to induce a senescent state.

More interestingly, after remaining in senescent state for 8-10 days, some of these cell were reactivated to generate dividing cells with a transformed phenotype. This phenomenon was seen in cells rendered senescent both by chromatin from sera of healthy subjects and from patients treated for cancer.

Apoptotic Chromatin Fragments Induce Apoptosis in Recipient Cells:

Apoptosis of recipient cells is detected by flowcytometry which reveals that apoptotic chromatin treatment results in varying degrees of apoptosis in different recipient cells. Apoptosis of a large proportion of NIH3T3 and MRC5 cells is seen after 48 hrs of treatment with apoptotic chromatin fragments purified from the serum of cancer patients previously treated with chemo- or radiotherapy. The apoptotic chromatin fragments purified from normal serum are far less effective in induction of apoptosis.

For lymphocyte apoptosis experiments, isolated lymphocytes from healthy subjects are treated with apoptotic chromatin fragments purified from the plasma/serum of patients with renal failure, diabetes, septicemia and cancer pretreated with chemo- or radiotherapy. Induction of apoptosis in a large proportion of lymphocytes is seen in all these conditions. However, when apoptotic chromatin fragments purified from the sera of healthy subjects are used only a negligible proportion of lymphocytes undergo apoptosis.

For analysis of apoptosis, propidium iodide stained cells are excited with 488 nm Argon laser and FL2(H) is recorded through >600 nm band pass filter on log scale. For analysis of Annexin V labeled cells, FL1 (FITC) emission is recorded through 530 nm band pass filter on log scale.

Apoptotic Chromatin Fragments Induce Oncogenic Transformation of Recipient Cells:

It is observed that apoptotic chromatin fragments purified from serum of cancer patients previously treated with chemo- or radiotherapy are capable of transforming recipient cells within 4-5 days. Apoptotic chromatin fragments purified from as little as 5-10 μl of serum have transforming activity. The application of higher quantities of apoptotic chromatin fragments purified from 25-100 μl of serum induce apoptosis and senescence of the recipient cells. As mentioned above, some of the senescent cells are reactivated after 8-10 days and generate dividing cells with a transformed phenotype. Therefore, it is evident that apoptotic chromatin fragments purified from sera of pre-treated cancer patients induce transformation by two mechanism: primarily, when added in low doses and secondarily, when added in higher doses following the reactivation of senescent cells.

On the other hand, apoptotic chromatin fragments purified from serum of healthy subjects are incapable of primary transformation of recipient cells even when added in high quantities viz. when purified from as much as 1000 μl of serum. However, repeated additions on alternate days of apoptotic chromatin fragments purified from 100 μl of serum from healthy subjects leads to secondary transformation of recipient cells following senescence as described above.

Transformed Cells are Tumorigenic in Nude Mice:

Several clones are developed from recipient cells that are transformed by apoptotic chromatin treatment as described above. These clones are injected into nude mice at a concentration of 5×10⁶ cells. Most injected clones form tumours within 2-4 weeks. Histological sections of tumours induced by NIH3T3 mouse fibroblast cells that are transformed with apoptotic chromatin fragments derived from Jurkat cells or sera of cancer patients are examined by FISH using human whole genomic probes. The presence of human DNA in these mouse tumours are clearly visible indicating further that human DNA/chromatin fragments get integrated into mouse cell genomes.

Loss of Biological Activity after Removal of Apoptotic Chromatin Fragments from Plasma:

It is observed that addition of plasma from patients with diabetes, renal failure, sepsis and pre-and post-treatment patients with cancer to lymphocytes isolated from healthy subjects induces apoptosis in a significant proportion of cells. The apoptosis inducing property of this plasma is virtually abolished when the above plasma fractions are subjected to immunoadsorption to remove apoptotic chromatin fragments. Since the induction of apoptosis is one of the biological end-points in a chain of pathological events that apoptotic chromatin fragments from serum bring about, it is obvious that removal of apoptotic chromatin fragments from blood by an ex vivo mechanism may act as method of treatment to retard or ameliorate the pathological consequences of diabetes, renal failure and sepsis as well as prevent the initiation and spread of cancer in the body.

### BRIEF DESCRIPTION OF THE DRAWINGS

FIG. 1: Physical characteristics of radioactively labeled chromatin fragments from apoptotic cultured cells as demonstrated by EM-autoradiography.

FIG. 2: Demonstration of immunopurified chromatin fragments from plasma by electron microscopy.

FIG. 3: Schematic diagram of the Cell Death Detection Elisa Kit used for the measurement of apoptotic chromatin fragments from plasma/serum.

FIG. 4: Plasma chromatin levels in healthy subjects and patients suffering from various diseases.

FIG. 5: Physical presence of radioactively labeled chromatin fragments derived from apoptotic pellet P1 from cultured donor cells within recipient cells, especially within their nuclei, as demonstrated by EM-autoradiography.

FIG. 6: Ingestion by healthy lymphocytes of apoptotic chromatin fragments immunopurified from plasma that were labeled by TUNEL method using Alexa conjugated dUTP.

FIG. 7: Integration of exogenous apoptotic chromatin fragments from cultured cells into genomes of recipients as demonstrated by Fluorescent In situ Hybridization (FISH).

FIG. 8: Integration of exogenous apoptotic chromatin fragments immunopurified from human serum into genomes of recipient cells as demonstrated by Fluorescent In situ Hybridization (FISH).

FIG. 9: DNA damage induced in various recipient cells by immunopurified chromatin fragments from serum as detected by antibody against phosphorylated γH2AX.

FIG. 10: Partial metaphases of recipient cells demonstrating chromosomal damage/abnormalities induced by treatment with immunopurified apoptotic chromatin fragments from serum.

FIG. 11: Chromosomal instability in the form of unusual centromeric amplifications induced in recipient cells by apoptotic P1 pellet from cultured donor cells as demonstrated by FISH.

FIG. 12: Inter simple-sequence repeat PCR showing genomic instability induced in recipient cells by immunopurified chromatin fragments from serum.

FIG. 13: Time course of development of aneuploidy in recipients after treatment with apoptotic P1 pellet from cultured donor cells as demonstrated by temporal flow cytometry.

FIG. 14: Induction of a senescent phenotype, persistent DNA damage and p53 upregulation in recipient cells induced by treatment with immunopurified apoptotic chromatin fragments from serum. Reactivation of senescent cells with generation of dividing progenies with a transformed phenotype are also shown.

FIG. 15: Induction of apoptosis of recipient cells by immunopurified apoptotic chromatin fragments from serum.

FIG. 16: Oncogenic transformation of recipient cells by immunopurified apoptotic chromatin fragments from serum.

FIG. 17: Oncogenically transformed cells growing in semi-solid medium.

FIG. 18: Oncogenically transformed cells form large tumours when injected subcutaneously into nude mice; and FISH showing presence of human DNA in tumours induced by injection of mouse cells transformed by apoptotic chromatin fragments immunopurified from sera of cancer patients.

FIG. 19: Induction of apoptosis in healthy lymphocytes by immunopurified apoptotic chromatin fragments from plasma of patients suffering from diabetes, renal failure, sepsis and cancer.

FIG. 20: Abolition of apoptosis-inducing activity of plasma from patients suffering from diabetes, renal failure, sepsis and cancer after removal of apoptotic chromatin fragments by immunoadsorption.

FIG. 21A: Flow diagram depicting the process for removal of apoptotic chromatin fragments from blood.

FIG. 21B: Flow diagram depicting the steps for removal of apoptotic chromatin by generation of CRP.

FIG. 21C: Flow diagram depicting the steps for removal of apoptotic chromatin by generation of PCRP.

FIG. 21D: Flow diagram depicting the steps for removal of apoptotic chromatin from PCRP by means of single or multiple chromatin removal chambers.

FIG. 22A: Sectional view of a rigid sedimentation chamber for generation of PCRP.

FIG. 22B: Sectional view of a flexible sedimentation chamber for generation of PCRP.

FIG. 22C: Sectional view of a specialized hollow fibre plasma filter for generation of PCRP.

FIG. 23A Sectional view of first chromatin removal chamber where separating means comprise matrix in the form of hollow fibres coated with appropriate reagents to adsorb chromatin.

FIG. 23B: Sectional view of first chromatin removal chamber where the separating means comprise matrix in the form of beads coated with appropriate reagents to adsorb chromatin.

FIG. 23C: Sectional view of the first chromatin removal chamber where density gradient centrifugation is carried out for selectively sedimenting large/dense chromatin fragments.

FIG. 23D: Sectional view of the first chromatin removal chamber where the separating means comprise flowcytometric cell sorter. The inset shows the cluster of platelets that are separated from PCRP.

FIG. 24: Sectional view of the third chromatin removal chamber where the separating means comprise high speed centrifugation

### DETAILED DESCRIPTION OF THE DRAWINGS

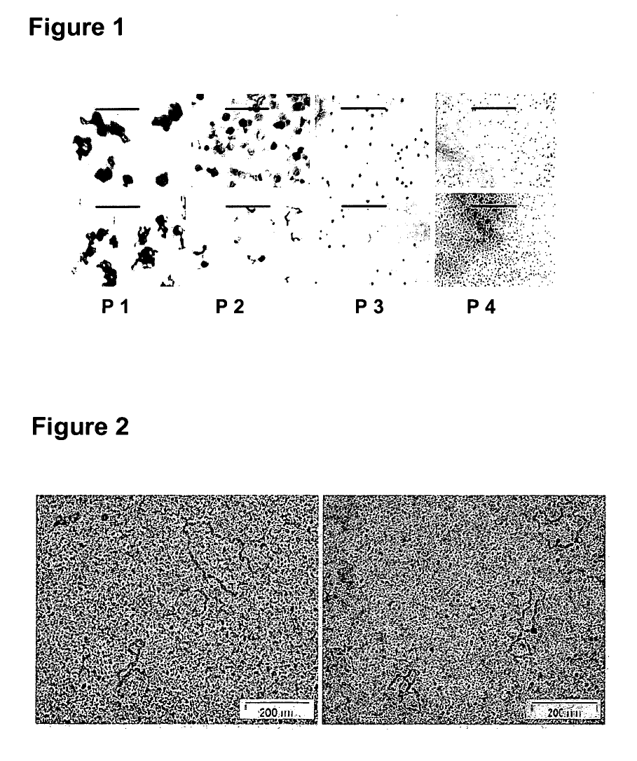

FIG. 1 shows physical characteristics of apoptotic chromatin fragments. EM-autoradiography of apoptotic chromatin fragments contained in pellets P1, P2, P3 and P4 derived from B16F10 cells that were pre-labeled with ³H-Thymidine (upper four panels), and ³⁵S-Methionine (lower four panels). Scale bars, 500 nm.

FIG. 2 shows physical characteristics as revealed by electron microscopy of apoptotic chromatin fragments immunopurified from plasma of cancer patients (left panel) and those immunopurified from plasma of normal subjects (right panel). Scale bars, 200 nmn.

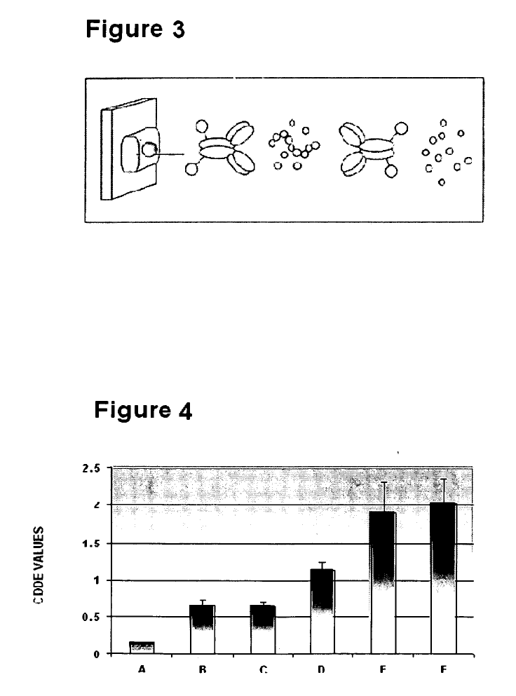

FIG. 3 shows the schematic diagram of the Cell Death Detection Elisa System used for measurement of apoptotic chromatin fragments in plasma/serum (downloaded from Roche Applied Sciences Homepage). In this system streptavidin coated polystyrene plates are used to which biotinylated antihistones antibodies bind. The free nucleosomes present in plasma/serum bind to the antihistone antibodies. The specificity of nucleosomes is determined by the binding of anti-DNA-antibody lebeled with peroxidase. The latter produces a colour reaction if nucleosomes i.e. molecules which contain both histones and DNA are present. The colour reaction is detected calorimetrically at 405 nm and the values are expressed as arbitery units.

FIG. 4 shows histrograms depicting mean (±SE) plasma levels of apoptotic chromatin fragments from normal subjects and those from patients suffering from various disease conditions A: healthy subjects (n=50); B: patients suffering from diabetes (n=30); C: patients suffering from cancer (n=50); D: same cancer patients treated with chemo- or radiotherapy (n=50); E: patients suffering from renal failure (n=30); F: Patients with sepsis (n=30).

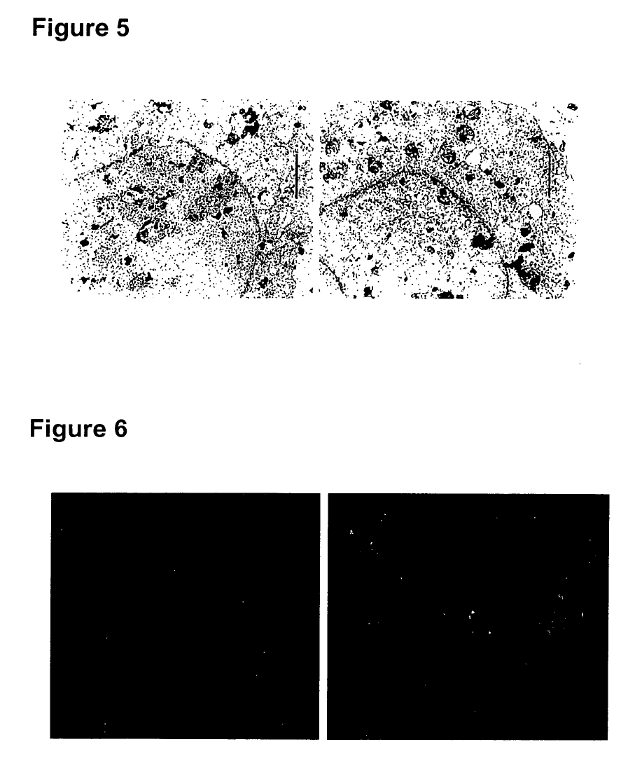

FIG. 5 shows physical presence of labeled chromatin fragments within the recipient cells especially within their nuclei. EM-autoradiography of sections of NIH3T3 cells treated for 72 hours with P1 (B16F10) labeled with ³H-Thymidine (left) and ³⁵S-Methionine (right). Scale bars, ˜500 nm.

FIG. 6 shows ingestion by lymphocytes of apoptotic chromatin fragments immunopurified from plasma of cancer patients labeled by TUNEL method using Alexa labeled 5′ dUTP. The nuclei are counterstained with DAPI. Normal lymphocytes (left panel) treated lymphocytes (right panel). The latter also shows that the lymphocytes have undergone apoptosis.

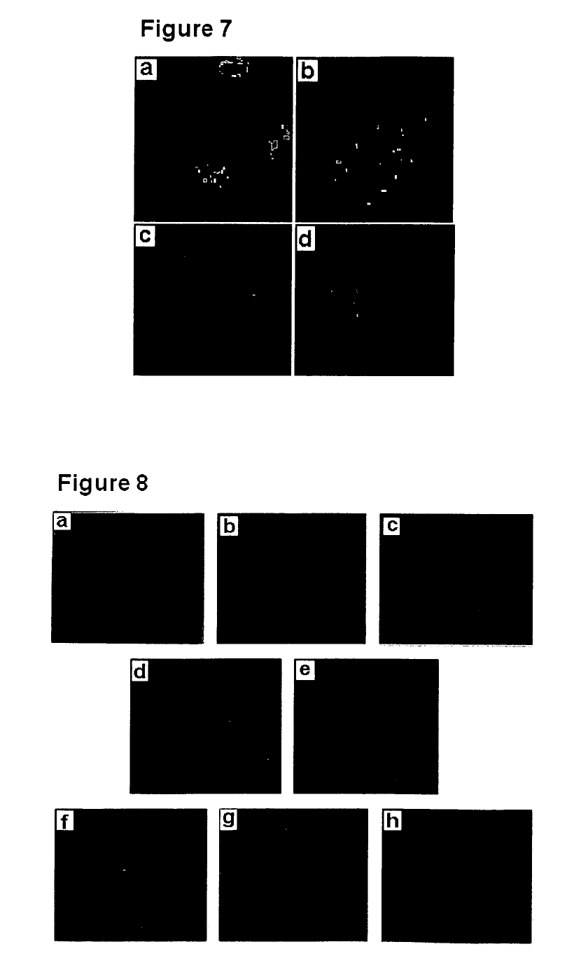

FIG. 7 shows the integration of exogenous apoptotic DNA fragments and centromeres derived from apoptotic cultured cells into genomes of recipients as detected by Fluorescent In situ Hybridization (FISH). NIH3T3 cells are treated with pellet P1 (Jurkat) for 48 hours and metaphase spreads are prepared after colcemid treatment. Fish is performed with human whole genomic painting probes or human pan-centromeric probes.
a) Integration of human DNA fragments derived from Jurkat cells in NIH3T3 mouse fibroblast interphase cells;
b) Integration of human DNA fragments derived from Jurkat cells in NIH3T3 mouse fibroblast metaphase cells;
c) Integration of human centromeres derived from Jurkat cells in mouse fibroblast interphase cells;
d) Integration of human centromeres derived from Jurkat cells in NIH3T3 mouse fibroblast metaphase cell.

FIG. 8 shows the integration of exogenous apoptotic DNA fragments and centromeres derived from sera of human subjects into genomes of recipients using Fluorescent In situ Hybridization (FISH). NIH3T3 mouse fibroblast, mouse ovary, mouse kidney and mouse liver cells are treated for 24 hours with apoptotic chromatin fragments immunopurified from sera of cancer patients and metaphase spreads are prepared after colcemid treatment. FISH is performed using human whole genomic and human pan-centromeric probes simultaneously.
a) NIH3T3 cells showing a conjoint human centromeric signal (Texas Red) together with a pericentromeric DNA signal (FITC).
b) The same fluorescent picture taken separately showing the FITC labeled DNA fragment.
c) The same fluorescent picture taken separately showing the Texas Red labeled centromere.
d) Mouse ovary cells showing a FITC labeled human DNA signal.
e) Mouse kidney cells showing a FITC labeled human DNA signal.
f) Mouse liver cells showing a conjoint human centromeric signal (Texas Red) together with a pericentromeric DNA signal (FITC).
g) The same fluorescent picture taken separately showing the FITC labeled DNA fragment.
h) The same fluorescent picture taken separately showing the Texas Red labeled centromere.

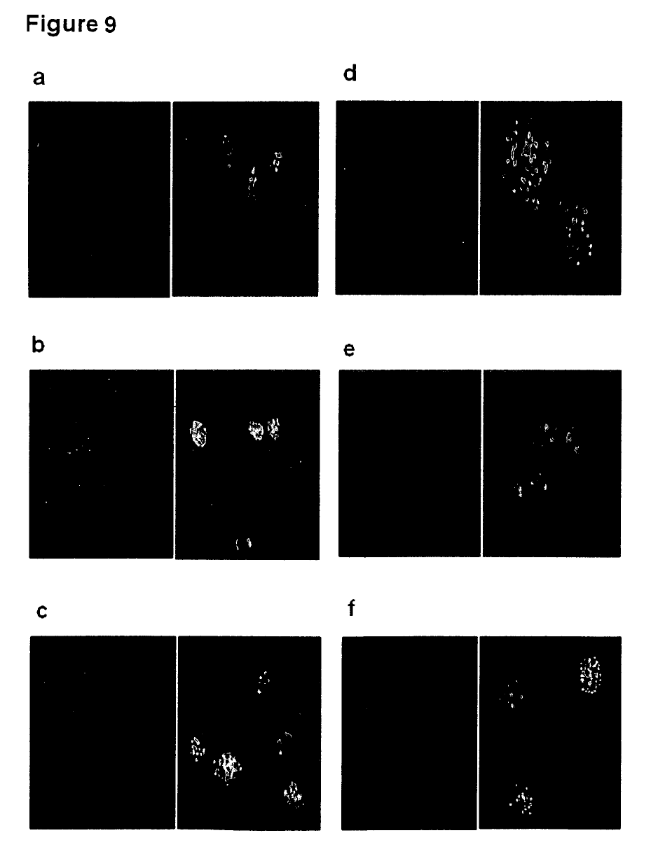

FIG. 9 shows DNA damage in various cells induced by treatment with immunopurified apoptotic chromatin fragments from sera of cancer patients treated with chemo- or radiotherapy. DNA damage induced after 24 hours of treatment is detected using a polyclonal antibody against phosphorylated γH2AX . Panels on the left are control cells and panels on the right are treated cells.
a) Mouse fibroblast cells
b) Mouse ovary cells
c) Mouse liver cells
d) MRC5 human fibroblast cells
e) Mouse kidney cells
f) Human neuronal cells

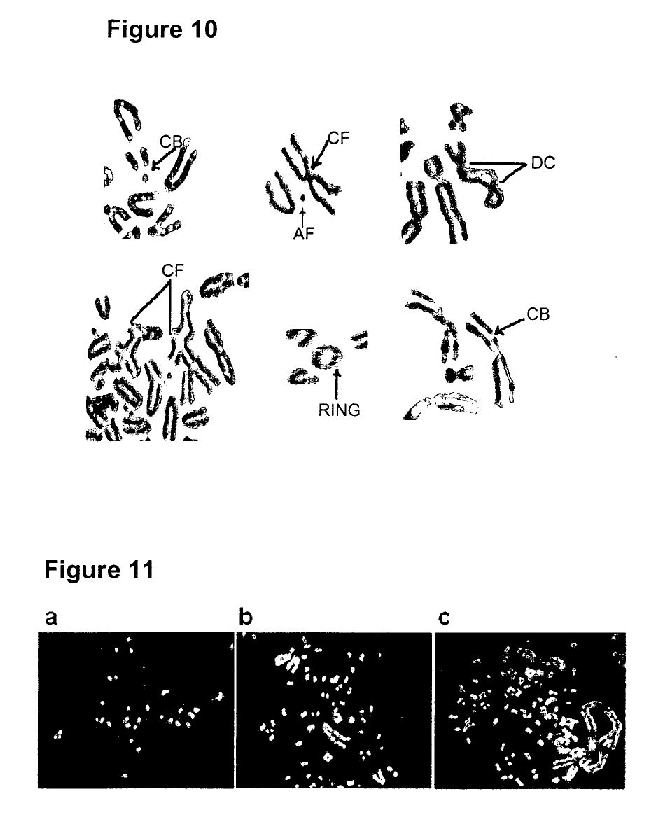

FIG. 10 shows partial metaphases of cells treated with immunopurified apoptotic chromatin fragments from sera of cancer patients. NIH3T3 cells are treated with immunopurified apoptotic chromatin fragments for 24 hours and metaphase spreads are prepared after colcemid treatment. Arrows point to different chromosomal abnormalities: AF=acrocentric fragment; CB=chromatid breakage; CF=centromeric fusion; DC=dicentric chromosomes; ring chromosomes.

FIG. 11 shows chromosomal instability in the form of centromeric amplifications induced by treatment with P1 pellet from apoptotic B16F1 cells.
a) FISH with a mouse pan-centromeric probe on metaphase spreads of normal NIH3T3 recipients;
b) and c) FISH with a mouse pan-centromeric probe on metaphase spreads of normal NIH3T3 recipients treated with P1 (B16F10) for 48 hours showing unusual centromeric amplifications.

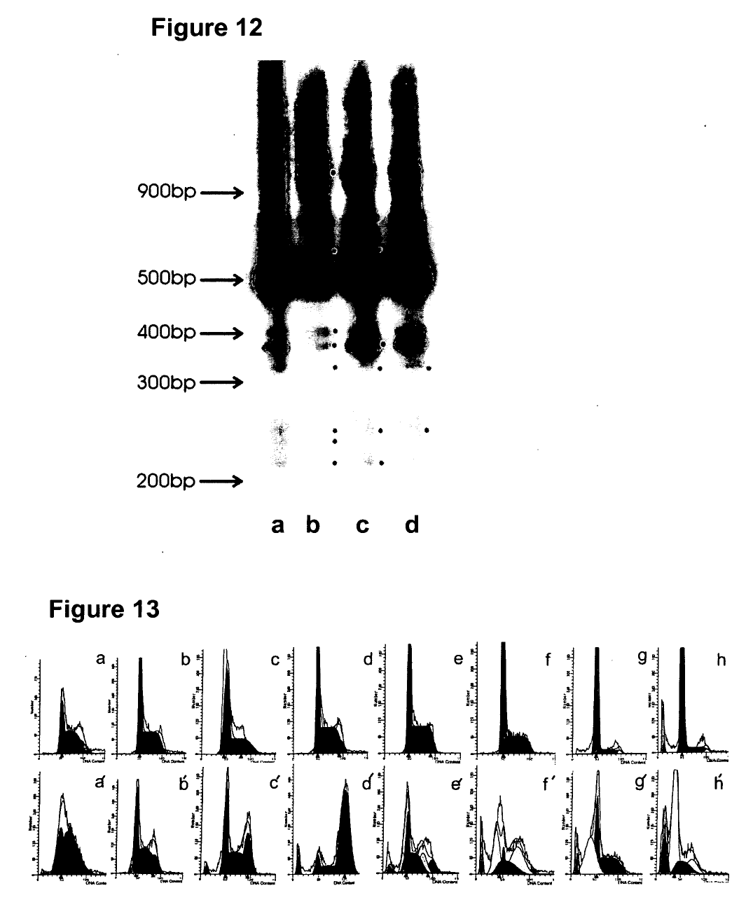

FIG. 12 shows genomic instability induced in NIH3T3 cells after 48 hours of treatment with immunopurified apoptotic chromatin fragments from sera of cancer patients treated with chemo- or radiotherapy. A primer (CA)₈ anchored with 5′-RG (where R is an equimolar mixture of adenosine and guanosine) is used for amplification of genomic DNA between the regions of CA repeats. Amplifications of deletions of various DNA bands are observed when the fragments are separated by PAGE and visualized by autoradiography.
a) Untreated NIH3T3 cells.
b) NIH3T3 cells treated with apoptotic chromatin fragments immunopurified from 25 μl of cancer serum.
c) NIH3T3 cells treated with apoptotic chromatin fragments immunopurified from 50 μl of cancer serum.
d) NIH3T3 cells treated with apoptotic chromatin fragments immunopurified from 100 μl of cancer serum.

FIG. 13 shows time course of development of aneuploidy in the recipient population after P1(B16F10) treatment. Temporal flowcytometric profiles of untreated NIH3T3 cells (a-h), and P1(B16F10) treated NIH3T3 cells(a′-h′). Sequential time points from left: 6, 12, 18, 24, 48, 72, 96, 120 hours respectively. Apoptotic cells are represented by the sub GI peaks.

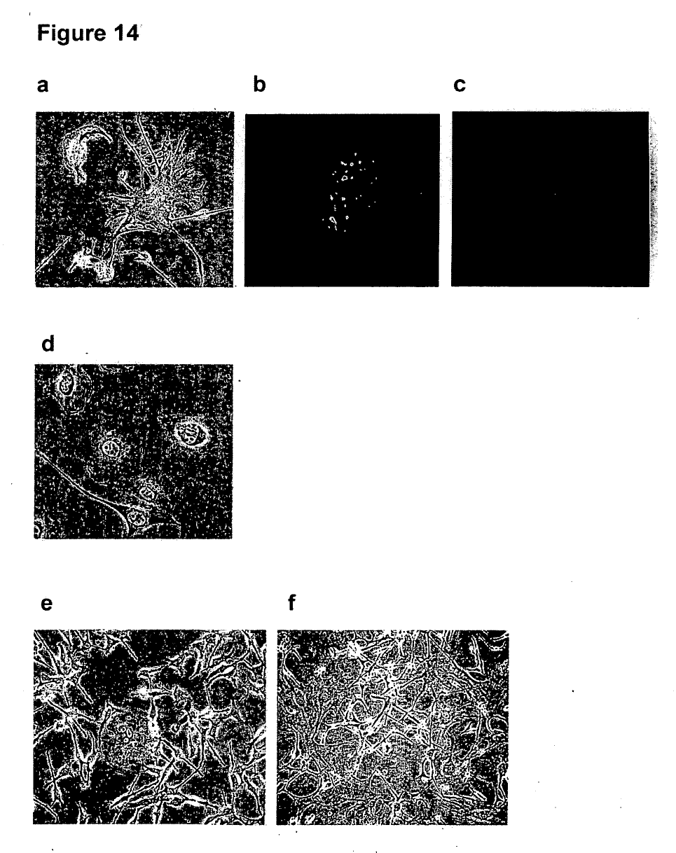

FIG. 14 shows induction of senescence of recipient cells treated with immunopurified chromatin fragments from patients treated for cancer and healthy subjects. The induction of senescence is assessed on the basis of i) cellular morphology; ii) persistence of DNA damage by immunodetection of phosphorelated γH2AX; iii) over expression of p53. For cancer serum, NIH3T3 cells are treated once with apoptotic DNA fragments purified from 100 μl of serum. Senescence is detected after 5-6 days. For serum from healthy subjects, NIH3T3 cells are treated repeatedly for 5 times every alternate day by apoptofic DNA fragment purified from 100 μl of serum.
a) Shows a senescent cell treated with apoptotic chromatin fragments immunopurified from cancer serum.
b) Shows persistent DNA damage in a senescent cell treated with apoptotic chromatin fragments immunopurified from cancer serum.
c) Shows p53 upregulation in a senescent cell treated with apoptotic chromatin fragments immunopurified from cancer serum.
d) Shows senescent cells treated repeatedly for 5 times every alternate day with apoptotic chromatin fragments immunopurified from normal serum.
e, f) Show reactivation of senescent cells to generate dividing cell with a transformed phenotype. (e) cells treated with apoptotic chromatin fragments immunopurified from cancer serum and (f) cells treated with apoptotic chromatin fragments immunopurified from normal serum.

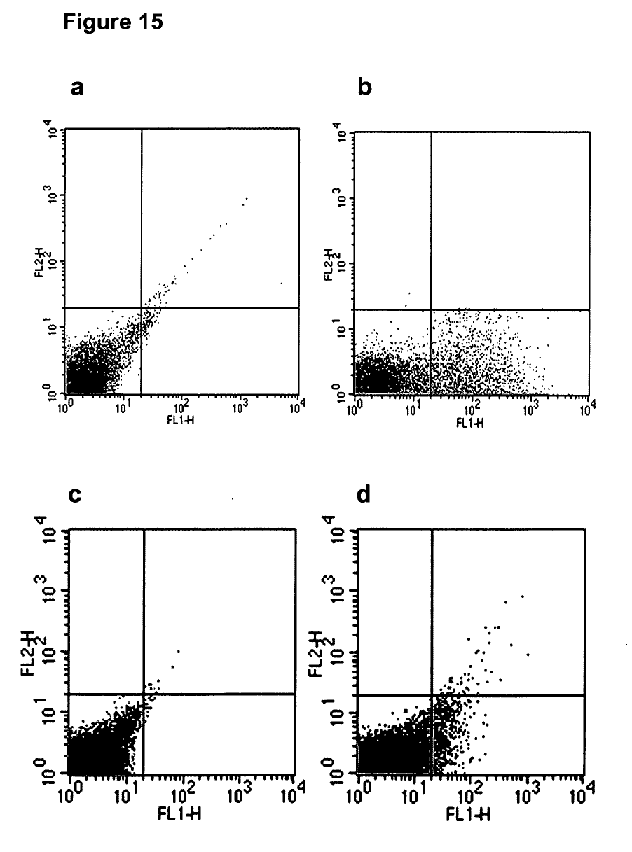

FIG. 15 shows induction of apoptosis after treatment of recipient cells for 48 hours with apoptotic chromatin fragments immunopurified from sera from cancer patients and healthy subjects. For detection of apoptosis, propidium iodide stained cells are excited with 488 nm Argon laser and FL2(H) is recorded through >600 nm band pass filter on log scale. For analysis of Annexin V labeled cells, FL1 (FITC) emission is recorded through 530 nm band pass filter on log scale.
a, b) NIH3T3 cells treated with apoptotic chromatin fragments immunopurified from normal sera (a) and sera from cancer patients (b).
c, d) MRC5 cells treated with apoptotic chromatin fragments immunopurified from normal sera (c) and sera from cancer patients (d).

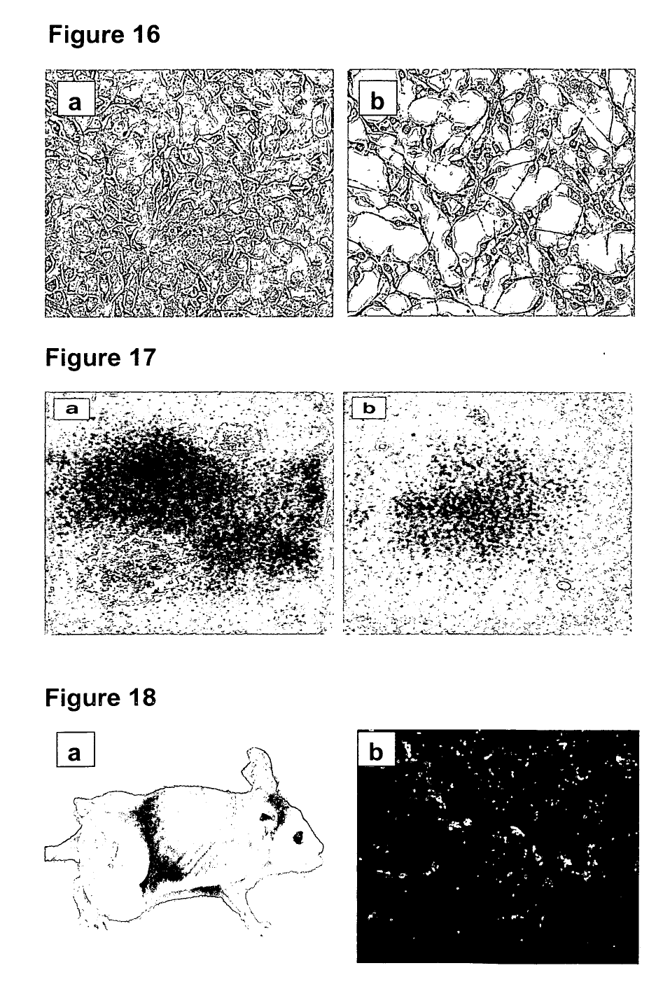

FIG. 16 shows oncogenic transformation of NIH3T3 cells by apoptotic chromatin fragments immunopurified from sera of cancer patients who were treated with chemo- or radiotherapy 24-48 hours earlier. Apoptotic chromatin fragments purified from 10 μl of cancer serum is added to 5×10⁴ NIH3T3 cells grown in 3 cm petri dishes. Transformation is seen after 4-5 days.
a) Normal NIH3T3 cells
b) Transformed NIH3T3 cells.

FIG. 17 shows growth of NIH3T3 cells transformed by apoptotic chromatin fragments immunopurified from cancer serum in soft agar. 10×10⁴ cells are seeded in 6 cm Petri dishes and colonies are observed after 2-3 weeks.
a) Transformed NIH3T3 cells form large colonies.
b) Normal NIH3T3 cells fail to form colonies.

FIG. 18 shows NIH3T3 cells transformed with apoptotic chromatin fragments immunopurified from cancer patients form tumours when injected subcutaneously into nude mice, and that these tumours contain human DNA.
a) 5×10⁶ cells are injected into nude mice and tumours are detected after 2-3 weeks.
b) Paraffin sections of these tumours are examined by FISH using human whole genomic probes. Presence of human DNA in mouse tumours is clearly seen.

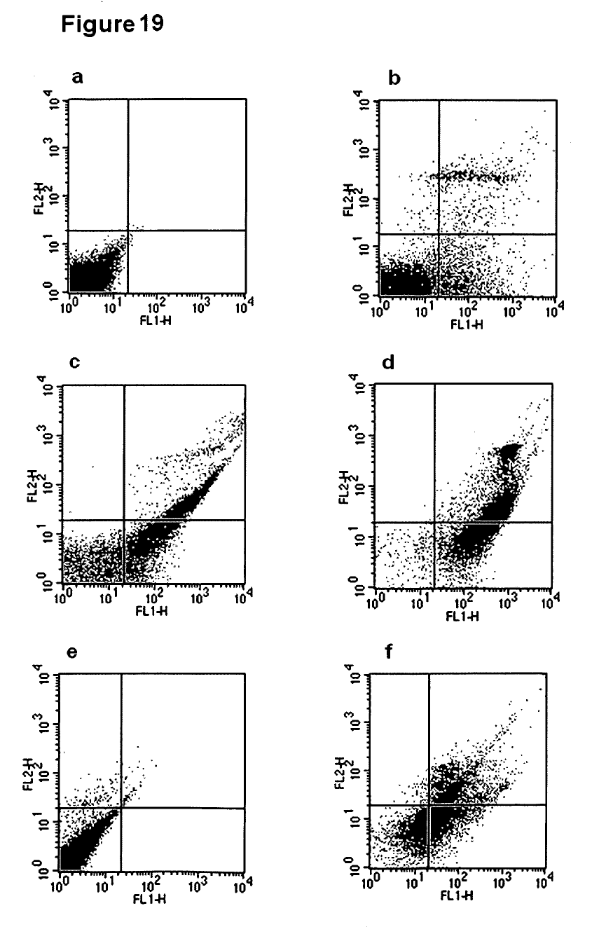

FIG. 19 shows induction of apoptosis in healthy lymphocytes by apoptotic chromatin fragments immunopurified from patients suffering form various diseases. Lymphocytes are isolated from healthy subjects and treated with appropriate immunopurified apoptotic chromatin fragments for 24 hours. Induction of apoptosis is assessed as described in FIG. 15.
a) Treatment of normal lymphocytes with apoptotic chromatin fragments immunopurified from plasma of normal subjects.
b) Treatment of normal lymphocytes with apoptotic chromatin fragments immunopurified from plasma of patients suffering from diabetes
c) Treatment of normal lymphocytes with apoptotic chromatin fragments immunopurified from plasma of patients with renal failure
d) Treatment of normal lymphocytes with apoptotic chromatin fragments immunopurified from plasma of patients with sepsis.
e) Treatment of normal lymphocytes with apoptotic chromatin fragments immunopurified from plasma of patients suffering from cancer prior to treatment.
f) Treatment of normal lymphocytes with apoptotic chromatin fragments immunopurified from plasma of patients suffering from cancer 24-48 hours after chemo- or radiotherapy.

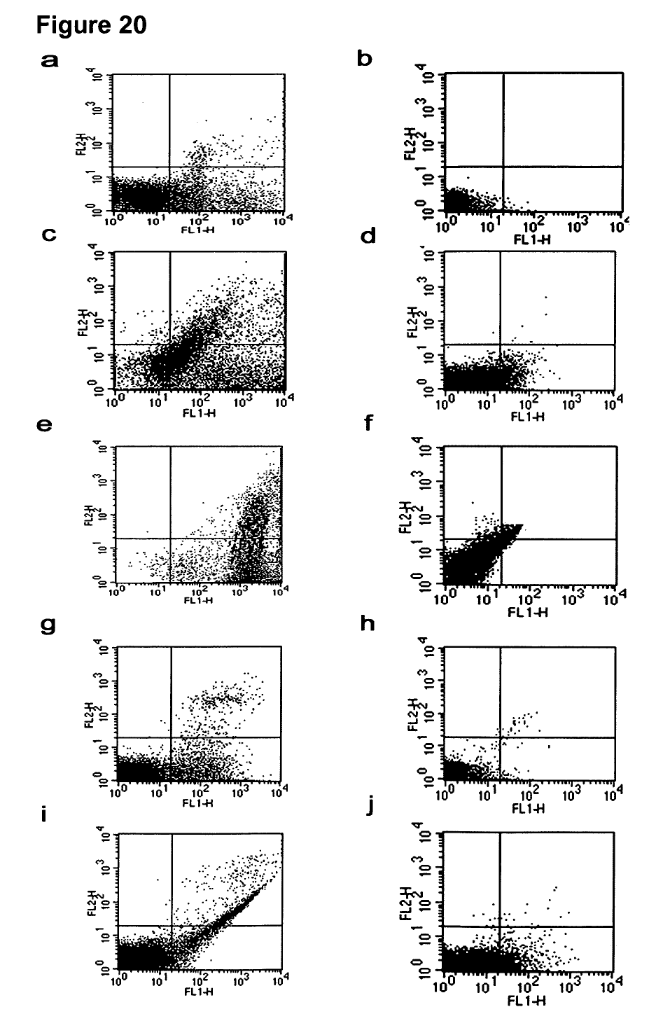

FIG. 20 shows loss of apoptosis-inducing activity of plasma after immunoadsorption of apoptotic chromatin fragments. Lymphocytes are isolated from healthy subjects and treated with plasma from patients suffering from different diseases or the same plasma after immunoadsorption of apoptotic chromatin fragments.
a, b) Lymphocytes treated with plasma from patients suffering from diabetes, before (a) and after immunoadsorption (b).
c, d) Lymphocytes treated with plasma from patients suffering from renal failure, before (c) and after immunoadsorption (d).
e, f) Lymphocytes treated with plasma from patients suffering from sepsis, before (e) and after immunoadsorption (f).
g, h) Lymphocytes treated with plasma from patients suffering from cancer, before (g) and after immunoadsorption (h).
i, j) Lymphocytes treated with plasma from patients suffering from cancer treated for 24-48 hours earlier with chemo- or radiotherapy, before (i) and after immunoadsorption (j).

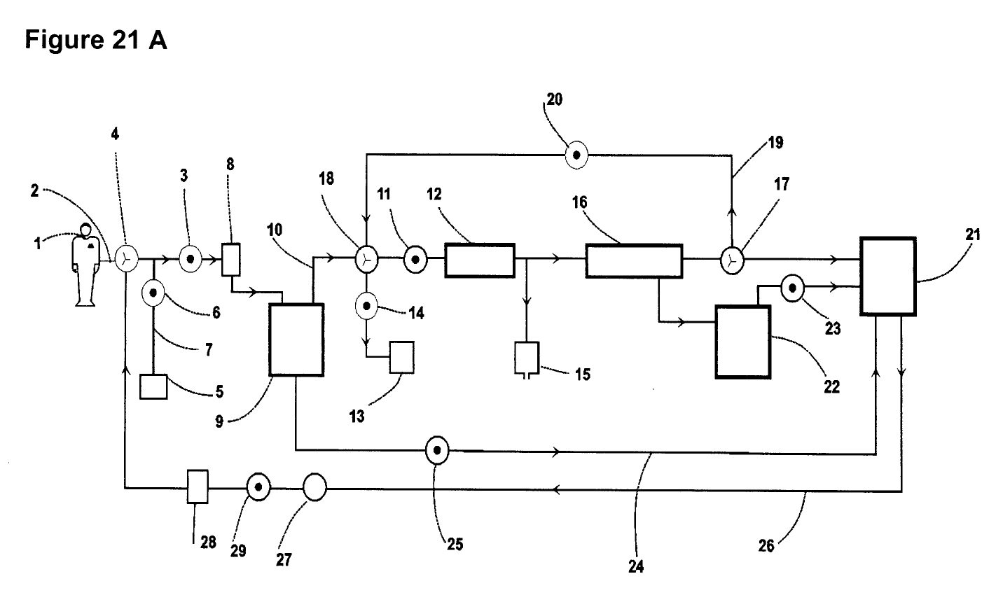

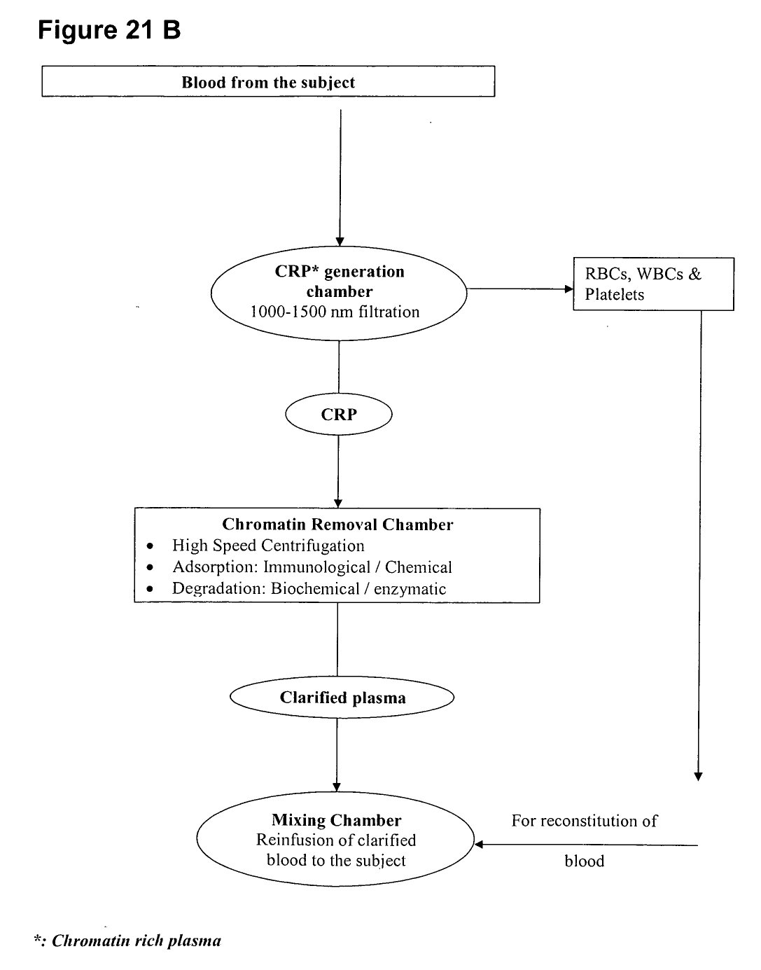

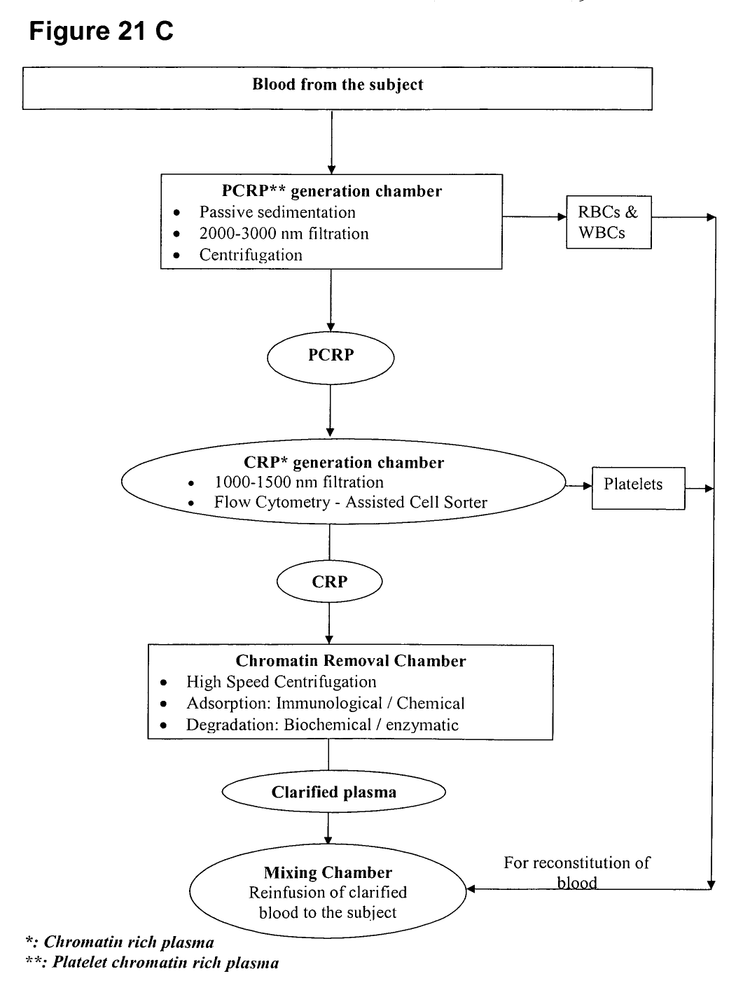

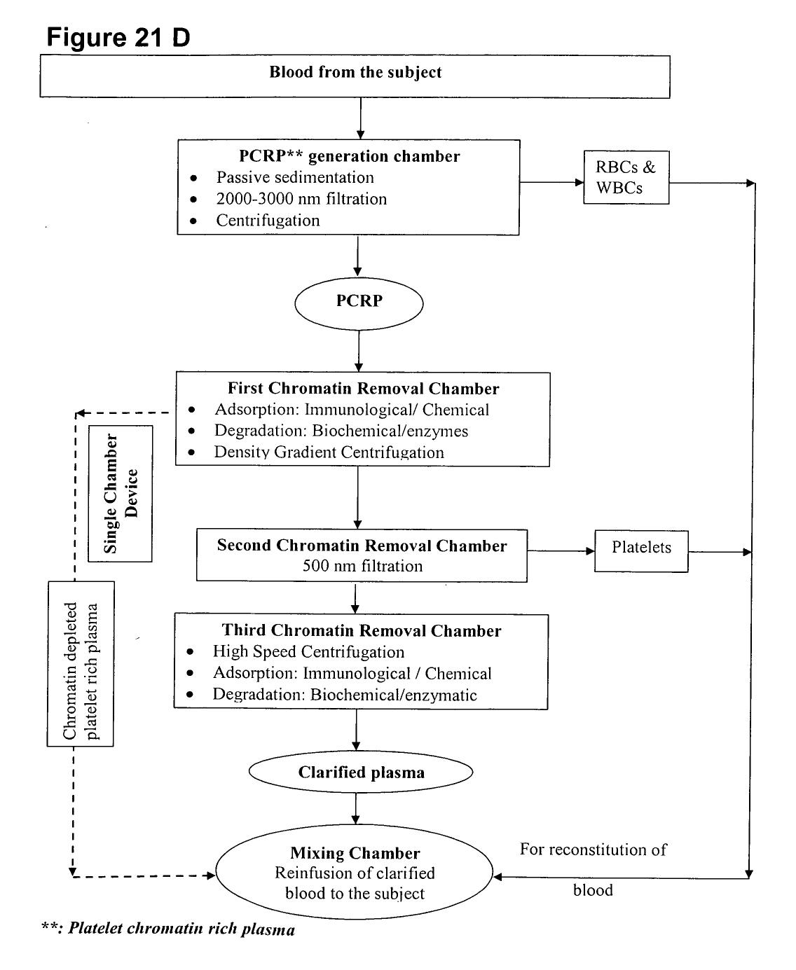

FIG. 21 A shows the flow diagram for removal of apoptotic chromatin fragments from blood according to a preferred embodiment. Blood from a suitable vein of the subject 1, enters the processing system via conduit 2. The blood is drawn from the subject by a peristaltic pump 3. The conduit is provided with a suitable three way valve 4 that can be set to control the direction of the flow of blood. Anti-coagulant is added from a reservoir 5 using an infusion pump 6 that communicates with the conduit 7. The anti-coagulated blood is led via an air trap 8 to PCRP generation chamber 9. The PCRP is drawn through an outflow conduit 10 using a peristaltic pump 11. This pump 11 propels the PCRP through the first chromatin removal chamber 12. In a preferred embodiment the first chromatin removal chamber is an immuno-adsorption device that removes chromatin from PCRP. An optional addition is the recharging/regeneration of the adsorption column. The latter is achieved by incorporating a reservoir 13 that contains appropriate chemical agent like hypertonic saline that can be passed through the column when it is not in use with the help of a peristaltic pump 14. The regenerating solution can then be drained into a container 15 before it is discarded. The chromatin depleted platelet rich plasma delivered from the first chromatin removal chamber 12 that has residual finer chromatin fragments is then flown through the second chromatin removal chamber 16 which is a ˜500 nm hollow fibre filtration device. The retentate from the filtration device is recirculated through the first chromatin removal chamber 12 and the filtration chamber 16 via a three-way valve 17 after the filtration chamber and another valve 18 before the first chromatin removal chamber. The recirculation loop uses conduit 19 and the flow through is propelled by a peristaltic pump 20. After the requisite number of recirculation cycles, the valve 17 directs the flow of the fraction of plasma with platelets but free of chromatin to the mixing chamber 21 for reconstitution with red and white cells that will eventually be reinfused to the subject. The filtrate plasma from the filtration chamber 16 is led to the third chromatin removal chamber 22 which in its preferred embodiment is a centrifugation chamber. The supernatant from this chamber containing clarified chromatin free plasma is then led into the mixing chamber 21 with the help of a peristaltic pump 23 for reconstitution with red and white cells and platelets for reinfusion to the subject. The mixing chamber 21 receives inputs from the retentate fraction from the second chromatin removal chamber 16, chromatin free plasma from the third chromatin removal chamber 22 and red and white cells from the PCRP generation chamber 9 via the conduit 24 propelled by the pump 25. The reconstituted blood is then removed by a conduit 26 and passed through a warmer 27 to bring the blood to body temperature and then reinfused to the subject via an air trap 28. The movement from the mixing chamber to the subject's vein is propelled by the peristatlic pump 29.

FIG. 21B shows flow diagram illustrating one aspect of the present invention wherein blood is treated to generate CRP by means of CRP generating means. Such means include filtration through membranes with porosity of ˜1000-˜1500 nm to separate RBCs, WBCs and platelets. The CRP is then conducted to the separating means which comprise the single/multiple chromatin removal chambers. Here apoptotic chromatin fragments are removed from CRP either by high speed centrifugation or adsorption: immunological/chemical or degradation: biochemical/enzymatic. The clarified plasma with the apoptotic chromatin fragments removed is then directed to the mixing chamber where it is mixed with RBCs, WBCs and platelets. The reconstituted blood is then directed back to the subject.

FIG. 21C shows flow diagram illustrating another aspect of the present invention wherein blood is treated to generate PCRP by means of PCRP generating means. Such means include passive sedimentation or tangential filtration using membranes of pore size ˜2000-˜3000 nm or centrifugation to separate RBCs and WBCs. The PCRP thus generated is then transmitted to the means for generating CRP which include filtration through membrane of porosity of ˜1000-˜1500 nm thereby separating the platelets. The CRP is then conducted to the separating means which comprise a chromatin removal chamber. Here apoptotic chromatin fragments are removed from CRP either by high speed centrifugation or adsorption: immunological/chemical or degradation: biochemical/enzymatic. The clarified plasma with the apoptotic chromatin removed is then directed to the mixing chamber where it is mixed with RBCs, WBCs and platelets. The reconstituted blood is then directed back to the subject.

FIG. 21D shows flow diagram illustrating another aspect of the present invention wherein separation of apoptotic chromatin from PCRP is achieved by means of single or multiple chromatin removal chambers. Blood from the subject is directed to PCRP generating means. Such means include passive sedimentation or tangential filtration using membranes with porosity of ˜2000-˜3000 nm or centrifugation to separate RBCs and WBCs. PCRP is then conducted to the separating means which comprise single/multiple chromatin removal chambers.//

For the system where single chromatin removal chamber is used PCRP is transmitted to the first chromatin removal chamber where the chromatin is removed either by adsorption: immunological/chemical or density gradient centrifugation or degradation: biochemical/enzymatic. Subsequently, the chromatin depleted platelet rich plasma is directed to the mixing chamber to be reconstituted with RBCs and WBCs for reinfusion to the subject.

For the system where multiple chromatin removal chambers are used, PCRP is first transmitted to the first chromatin removal chamber where the chromatin fragments are removed either by adsorption: immunological/chemical or density gradient centrifugation or degradation: biochemical/enzymatic. It is then directed to the second chromatin removal chamber where filtration through membranes with a porosity of ˜500 nm is done to remove platelets and then to the third chromatin removal chamber. In this chamber further chromatin is removed either by high speed centrifugation or adsorption: immunological/chemical or degradation: biochemical/enzymatic. The clarified plasma from this chamber is directed to the mixing chamber where it is mixed with RBCs, WBCs and platelets. The reconstituted blood is then directed back to the subject.

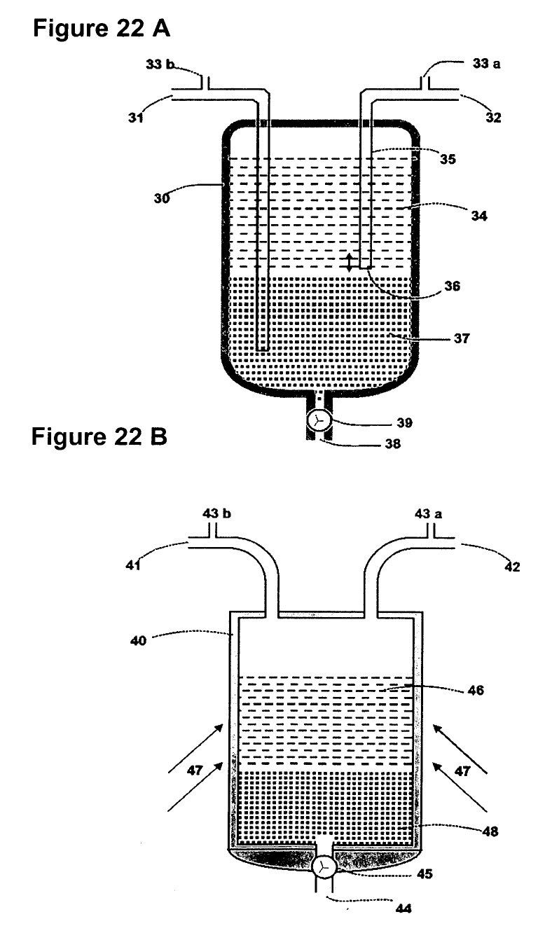

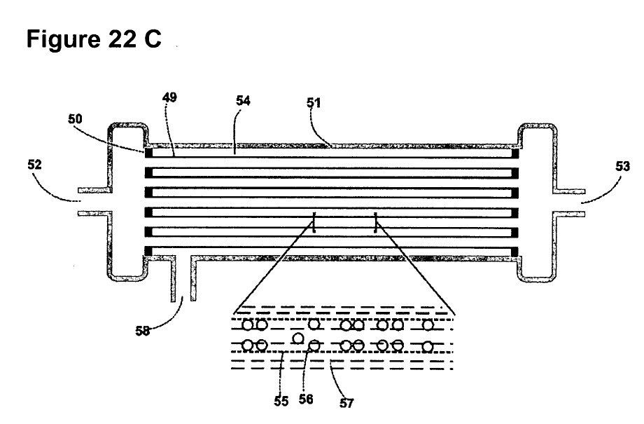

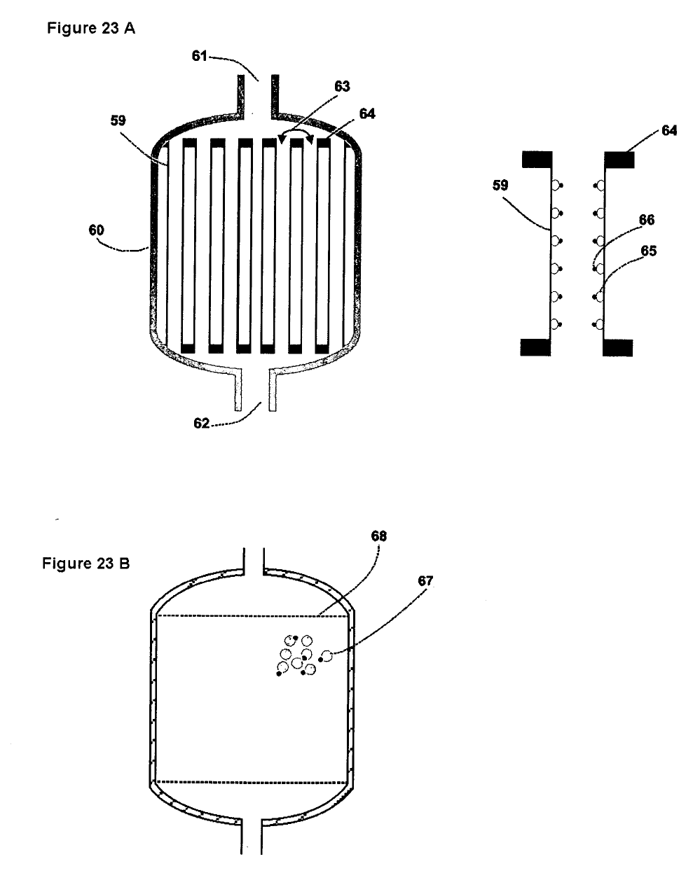

FIG. 22A: This figure shows the sectional view of the sedimentation chamber for generation of PCRP from whole blood. It is a cylindrical container 30 having an inlet 31 and outlet 32 each having sampling/injection ports for collecting samples for measuring chromatin levels 33a and infusing additives like anticoagulant 33b. The inlet delivers the blood from the subject and after the process of passive sedimentation the PCRP in the form of supernatant 34 is removed by the outlet conduit 35. The conduit 35 is so designed that lower end of the conduit 36 can be adjusted to position it within the chamber just above the upper level of sedimented red and white cells 37. Further, the chamber is provided with a drain outlet 38 at the bottom with appropriate conduit and a valve 39 to deliver the sedimented red and white blood cells to the mixing chamber for reconstitution of blood with clarified plasma and platelets at the end of the process.

FIG. 22B: This figure shows the sectional view of another embodiment of the sedimentation chamber for generation of PCRP from whole blood wherein the chamber is made of flexible plastic like plasticised polyvinylchloride (PVC) and the like. It has a housing 40 with an inlet 41, outlet 42 and a drain port 44 with appropriate conduits attached. The inlet and outlet conduits have sampling/injection ports for measuring chromatin levels 43a and for infusing additives like anticoagulants 43b. The drain port 44 has a valve 45. The flexible nature of the housing allows for the delivery of the supernatant PCRP 46 through the outlet 42 by graduated extrinsic compression 47 and the same could be done for delivery of sediment red and white blood cells 48 through the drain port 44 for reconstitution of blood at the end of the process.

FIG. 22C: This figure shows the sectional view of the specialized hollow fibre plasmafilter used for generation of PCRP. It comprises of porous membranes in the form of hollow fibres 49 cemented with the help of a potting compound 50 at the two ends of the housing 51. The housing has an inlet 52 for entry of whole blood into the hollow fibres, an outlet 53 for outflow of retentate with blood cells. The space around the hollow fibres in the housing is the filtrate chamber 54. The membrane is specifically designed such that the pores 55 are between ˜2000-˜3000 nm in diameter to retain the red and white blood cells 56 and to allow the PCRP 57 to be filtered out in the filtrate chamber 54 of the housing. The filtrate thus obtained is removed from a collection port 58 provided in the filtrate chamber 54.

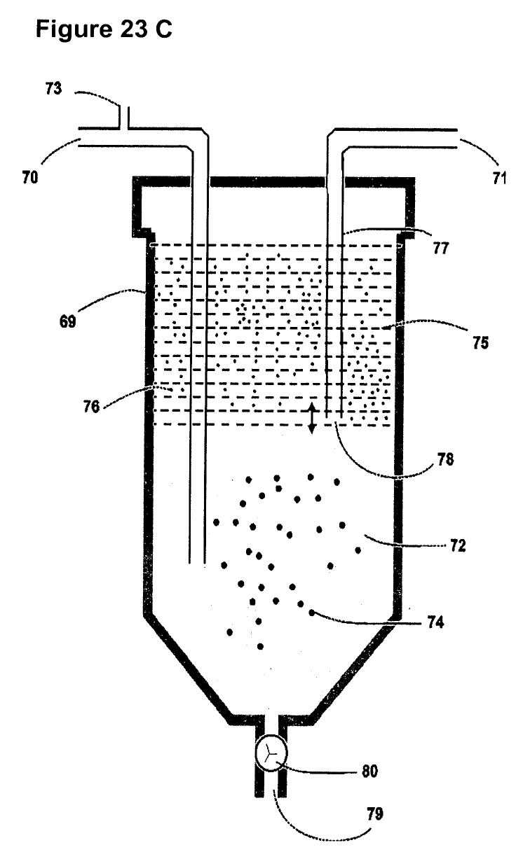

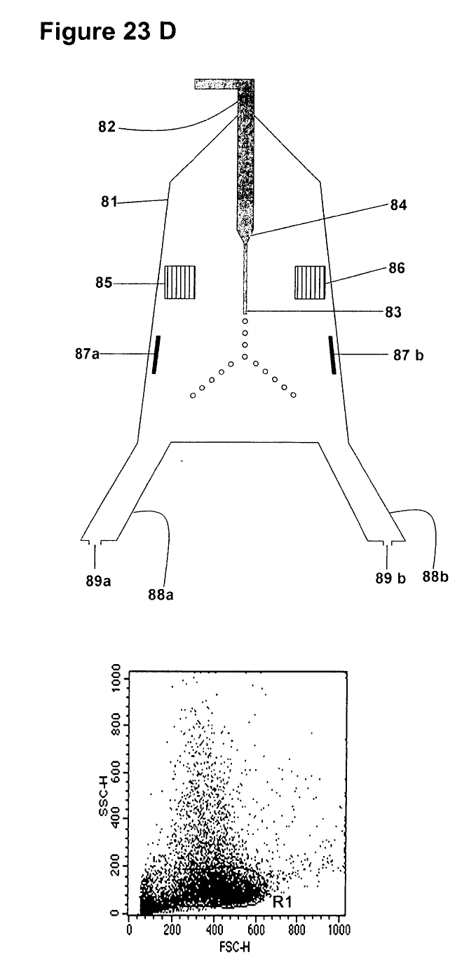

FIG. 23A shows a sectional view of the first chromatin removal chamber where the separating means comprise matrix in the form of hollow fibres 59 coated with appropriate reagents such as antibodies or cationic moieties or biochemical agents. The housing 60 for these coated hollow fibres has an inlet 61 and outlet 62. The PCRP enters the lumen of hollow fibres 63. The two ends of hollow fibres are cemented with the help of a potting compound 64 to exclude the space between the fibres from communicating with the PCRP. The inner surface of hollow fibres is coated with appropriate reagent 65 that binds or adsorbs or degrades the chromatin fragments 66.

FIG. 23B shows a sectional view of the first chromatin removal chamber where the separating means comprises matrix in the form of beads 67 coated with appropriate reagents. The basic structure of the device is the same as one shown in FIG. 23A except that the matrix is in the form of beads coated with appropriate reagents 67 that are retained within the device by a limiting membrane or mesh 68 that allows PCRP to flow through.

FIG. 23C shows a sectional view of the first chromatin removal chamber wherein the separation of chromatin from PCRP is achieved by density gradient centrifugation. The chamber consists of a housing 69 having an inlet 70 for entry of PCRP and an outlet 71 for the delivery of clarified plasma. The inlet 70 and outlet 71 are connected to flexible channels/tubes that pass through caps/lids that allow free rotation of the main chamber without compromising physical entity and sterility. The density gradient for centrifugation is created by using a suitable medium 72 which is introduced into the chamber by a side-port 73 on the inlet. A motorized rotor drives the rotation of the chamber at an appropriate speed and the denser chromatin fragments 74 sediment in the density gradient medium with a minimal loss of platelets. The supernatant 75, which is chromatin depleted platelet rich plasma which has residual finer chromatin fragments 76, is delivered via the outlet conduit 77. The lower end of this outlet conduit 78 is adjustable to a height just above the density gradient medium. There is a drain at the bottom 79 with a valve 80 that can then be used to discard the used up density gradient medium.

FIG. 23D shows a sectional view of a flowcytometric cell sorter. The device has a flow cell that has a housing 81 where the PCRP is delivered by a conduit 82 and is converted into a thin laminar stream 83 after it is released from a nozzle 84. The flow cell has laser source 85 and a photocell 86 for detecting scattered light. A pair of plates 87a and 87b with electrostatic charge are also placed along the length of PCRP movement. With the help of electrostatic, physical or fluorescent properties the thin stream of PCRP is segregated into the two streams one containing chromatin and the other containing platelets that are collected in separate receptacles 88a and 88b. Each of these receptacles has an outlet 89a and 89b . The platelets are returned to the subject and the plasma containing apoptotic chromatin fragments is processed for removal of such apoptotic fragments. The inset shows the platelet cluster when PCRP is analyzed by a flowsorter.

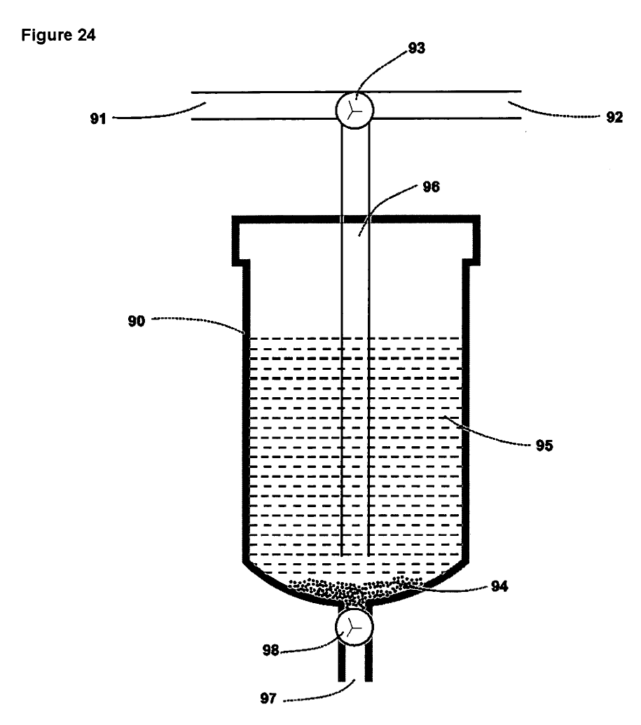

FIG. 24 shows a sectional view of the third chromatin removal chamber wherein the separation of finer/lighter chromatin fragments from platelet free plasma generated in the filtrate from second chromatin removal chamber (such as a standard hollow fibre plasma filter, not shown) is achieved by high speed centrifugation. The centrifugation is carried out in one or more containers. The container has a housing 90 with an inlet 91 and outlet 92 connected to suitable conduits. A valve 93 regulates the flow in and out of the container. The container rotates at appropriate speed that leads to the sedimentation of finer chromatin fragments 94 and the supernatant, which is chromatin free plasma 95 is delivered via the conduit 96. The sedimented chromatin is rejected by using the drain 97 that has a valve 98.

### Advantages of the Invention

The advantages of the present invention reside in the fact that it can prevent, ameliorate or retard the initiation or progression of all pathological phenomena that are associated with increased apoptotic turnover and are caused by DNA damage to healthy cells in the body by chromatin fragments derived from apoptotic cells carried in circulation that may lead to initiation and spread of cancer; ageing and age related diseases such as Alzheimer's disease, Parkinson's disease, stroke, atherovascular diseases, diabetes; renal failure; inflammation, severe infections, sepsis syndrome, multiorgan failure; autoimmune disorders; HIV/AIDS; spread of viral infections in the body as well as the harmful effects of transfusion of blood or blood products. More specifically, the advantages of the invention are:
i. The method of the invention is for removal of apoptotic chromatin fragments from blood by ex vivo purification of blood to treat/prevent initiation or progression of various pathological conditions.
ii. Such ex-vivo purification to rid circulating blood of harmful apoptotic chromatin fragments is the basis for treatment to include prevention of initiation and spread of cancer within the body.
iii. Such ex-vivo purification to rid circulating blood of harmful apoptotic chromatin fragments is the basis for treatment to include prevention or retardation of the process of ageing and age related diseases such as Alzheimer's disease, Parkinson's disease, stroke, atherovascular diseases, diabetes etc.
iv. Such ex-vivo purification to rid circulating blood of harmful apoptotic chromatin fragments is the basis for treatment to include prevention or retardation of all diseases associated with increased apoptosis such as renal failure; inflammation, severe infections, septic shock, multiorgan failure, autoimmune diseases etc.
v. Such ex-vivo purification to rid circulating blood of harmful apoptotic chromatin fragments is the basis for treatment to include prevention or amelioration of HIV/AIDS and the spread of viral infections within the body.
vi. Such ex-vivo purification to rid donor blood or blood products of harmful apoptotic chromatin fragments before transfusion is the basis for treatment to include prevention of the harmful effects of exogenous chromatin load on the recipient.

1. A method of prevention/treatment of pathological consequences of DNA damage triggered by incorporation of circulating apoptotic chromatin fragments into healthy cells of individuals/patients in need therefore, said method comprising ex vivo or extra corporeal treatment of blood/plasma for removal of circulating chromatin fragments released from apoptotic cells which apoptotic chromatin fragments are capable of triggering DNA damage leading to genomic instability, senescence, apoptosis and cancerous transformation of healthy cells on being integrated into their genomes

2. A method of treatment as claimed in claim 1 comprising ex vivo or extra corporeal treatment of blood to rid circulating blood of apoptotic chromatin fragments for prevention of initiation and spread of cancer within the body.

3. A method of treatment as claimed in claim 1 comprising ex-vivo treatment of blood to rid circulating blood of apoptotic chromatin for prevention or retardation of the process of ageing and age related diseases such as Alzheimer's disease, Parkinson's disease, stroke, atherovascular diseases, diabetes etc.

4. A method of treatment as claimed in claim 1 comprising ex-vivo treatment of blood to rid circulating blood of apoptotic chromatin fragments for prevention or retardation of diseases associated with increased apoptosis selected from renal failure; inflammation, severe infections, septic shock, multiorgan failure, autoimmune diseases and the like.

5. A method of treatment as claimed in claim 1 comprising ex-vivo treatment of blood to rid circulating blood of apoptotic chromatin fragments for prevention or amelioration of HIV/AIDS and the spread of viral infections within the body.

6. A method of treatment as claimed in claim 1 comprising ex-vivo treatment of blood to rid donor blood or blood products of apoptotic chromatin fragments before transfusion for prevention of the harmful effects of exogenous chromatin load on the recipient and its adverse effects.

7. A method of treatment as claimed in claim 1 wherein the ex vivo or extra corporeal treatment of blood comprises transmitting blood from body to means for removal of blood cells therefrom; further transferring apoptotic chromatin rich plasma (CRP) or platelets and apoptotic chromatin rich plasma (PCRP) to separating means comprising single and/or multiple chromatin removal chamber(s) wherein apoptotic chromatin are removed from CRP or PCRP; optionally recycling the treated PCRP through single chamber or first and second chamber (for multiple chamber system) through operation of valves; further transferring said treated CRP or PCRP devoid of apoptotic chromatin to means for reconstitution of blood and transmitting the blood cells from the means for separation of the blood cells to the means for reconstitution; finally, transmitting reconstituted treated blood to body; maintaining direction of blood and its flow rate by valves and pumps at appropriate position and maintaining compatible body temperature; monitoring efficiency of removal of apoptotic chromatin from CRP or PCRP by sampling ports.

8. A method of treatment as claimed in claim 7 wherein the ex vivo or extra corporeal treatment of blood comprises passing the blood through a system comprising:
means adapted for removal of apoptotic chromatin rich plasma (CRP) from red and white blood cells and platelets;
separating means adapted to remove apoptotic chromatin from CRP;
means adapted to reconstitute blood after removal of the apoptotic chromatin;
means adapted to communicating and guiding of the blood or plasma to the said separating means through the means for removal of CRP from blood cells, to means to reconstitute blood and direct the treated blood back into the body

9. A method of treatment as claimed in claim 7 wherein the ex vivo or extra corporeal treatment of blood comprises passing the blood through a system comprising: means adapted for removal of platelets and apoptotic chromatin rich plasma (PCRP) from red and white blood cells;
means adapted for removal of apoptotic chromatin rich plasma (CRP) from PCRP;
separating means adapted to remove apoptotic chromatin from PCRP;
means adapted to reconstitute blood after removal of the apoptotic chromatin;
means adapted to communicating and guiding of the blood or plasma to the said separating means through the means for removal of PCRP from blood cells, to means to reconstitute blood and direct the treated blood back into the body.

10. A method of treatment as claimed in claim 8 wherein means adapted for separation of RBCs and WBCs and platelets from CRP in the system through which blood is passed comprises of filtration system having membrane of appropriate porosity of ˜1000-˜1500 nm in the form of hollow fibres or sheets.

11. A method of treatment as claimed in claim 10 wherein said filtration membrane and hollow fibres are placed in a housing with inlet for entry of whole blood and outlet for outflow of retentate with blood cells, the space in the housing acting as filtrate chamber where CRP is collected and the CRP thus obtained being removed from collection port provided in the filtrate chamber, leading to the production of the filtrate (CRP) from whole blood.

12. A method of treatment as claimed in claim 9 wherein means adapted for separation of blood cells (RBCs and WBCs) from PCRP in the system through which blood is passed is selected from the group consisting of sedimentation chamber wherein blood from the body is separated into PCRP and blood cells by passive sedimentation, filtration system having membrane of appropriate porosity of ˜2000 to ˜3000 nm for tangential filtration and selective contraption adapted for the sedimentation of RBCs and WBCs from PCRP such as a centrifuge.

13. A method of treatment as claimed in claim 12 wherein for generation of PCRP from red and white blood cells the blood is passed in the said system through sedimentation chamber with one or more containers wherein RBCs and WBCs but not platelets and chromatin undergo passive sedimentation such that each container has an inlet and an outlet connected to suitable conduits wherein outlet delivers the supernatant namely PCRP.

14. A method of treatment as claimed in claim 12 wherein for generation of PCRP from red and white blood cells the blood is passed in the said system through said filtration membranes placed in a housing preferably, with inlet for entry of whole blood and outlet for outflow of retentate with blood cells, the space in the housing acting as filtrate chamber where PCRP is collected and the PCRP thus obtained is removed from collection port provided in the filtrate chamber, leading to the production of the filtrate (PCRP) from whole blood.

15. A method of treatment as claimed in claim 12 wherein for generation of PCRP from red and white blood cells the blood is passed in the said system through centrifugation chamber with one or more containers with appropriate rotor to sediment red and white cells but not the chromatin fragments and platelets, and each container has an inlet and an outlet connected to suitable conduits wherein the outlet delivers the supernatant namely the PCRP.

16. A method of treatment as claimed in claim 8 wherein removal of CRP from PCRP is carried out in the said system by filtration system having membrane of appropriate porosity for tangential filtration.

17. A method of treatment as claimed in claim 8 wherein removal of CRP from PCRP is carried out in the said system by flowcytometry-assisted cell sorter which separates platelets from PCRP thus generating CRP.

18. A method of treatment as claimed in claim 1 wherein blood is passed in the said system through the separating means comprising selectively single or multiple chromatin removal chamber(s).

19. A method of treatment as claimed in claim 18 wherein the removal of apoptotic chromatin fragments from CRP in the chromatin removal chamber is carried out selectively by high speed centrifugation of CRP to precipitate chromatin fragments or by using chemical agents, and/or immunological agents/antibodies, and/or biochemical agents/enzymes coated on matrices.

20. A system as claimed in claim 18 wherein removal of large/dense apoptotic chromatin from PCRP in the chromatin removal chamber (s) is carried out selectively by chemical agents, immunological agents/antibodies, biochemical agents/enzymes coated on matrices, contraption adapted for density gradient centrifugation of PCRP to precipitate chromatin fragments.

21. A method of treatment as claimed in claim 18 wherein removal of fine chromatin from the chromatin depleted platelet rich blood or plasma in multiple chromatin removal chambers comprise passing the blood through first chromatin removal chamber comprising selectively chemical agents, immunological agents/antibodies biochemical agents/enzymes coated on matrices, contraption adapted for density gradient centrifugation of PCRP to precipitate chromatin fragments, followed by passing through second chromatin removal chamber comprising filtration means comprising filtration membrane of appropriate porosity such that the platelets are retained and the fine chromatin fragments and plasma are filtered out- and additionally passed through a third chromatin removal chamber(s) for the removal of finer chromatin fragments from the chromatin depleted platelet free plasmaselectively by high speed centrifugation at appropriate centrifugal force to sediment the finer chromatin and/or immunological agents/antibodies, and/or chemical agents, and/or biochemical agents/enzymes coated on matrices.

22. A method of treatment as claimed in claim 7 wherein after removal of apoptotic chromatin reconstitution of blood occurs in means for reconstitution of blood, which comprises suitable mixing chamber wherein the CRP or PCRP rendered free of apoptotic chromatin by single and/or multiple chambers separating means is mixed with the blood cells and platelets.

23. A method of treatment as claimed in claim 7 wherein blood or plasma is communicated from body to the means for generating CRP/PCRP, separating means and the reconstitution chamber(s) and treated blood back to the body by means for communication comprising of suitable conduits with catheter.

24. A method of treatment as claimed in claim 19 wherein the said chemical agent is an ion-exchange resin in an anion-exchange column of DEAE—sephadex and the like.

25. A method of treatment as claimed in claim 19 wherein the said immunological agent is antibodies.

26. A method of treatment as claimed in claim 19 wherein the said biochemical/enzymatic agents are selected from DNAase, Nucleases and the like.

27. A method of treatment as claimed in claim 7 wherein the said monitoring of efficiency of removal of apoptotic chromatin from CRP or PCRP by sampling ports is carried out by being connecting to appropriate commercially available assay kit adapted to determine the content of chromatin fragments at the said inlet and outlet.
